(19) United States
(12) Patent Application Publication
(10) Pub. No.: US 2024/0042162 A1
(43) Pub. Date: Feb. 8, 2024

(54) WEARABLE DEVICE FOR VIBROTACTILE AND/OR THERMAL NERVE STIMULATION

(71) Applicant: Bose Corporation, Framingham, MA (US)

(72) Inventors: Adam Charles Furman, Bolton, MA (US); Keith Dana Martin, Westborough, MA (US); Christian Alan Trott, Charleston, SC (US); John Andrew Trotter, Westborough, MA (US); Leonard Varghese, South Dartmouth, MA (US)

(21) Appl. No.: 18/266,107

(22) PCT Filed: Dec. 6, 2021
(86) PCT No.: PCT/US2021/062002

§ 371 Date: Jun. 8, 2023

Related U.S. Application Data
(60) Provisional application No. 63/122,604, filed on Dec. 8, 2020.

(51) Int. Cl. A61M 21/00

(52) U.S. Cl. CPC A61M 21/00; H04R 1/1016

(57) ABSTRACT

Various aspects include wearable devices and related approaches for stimulating nerves proximate a user's ear, e.g., providing one or more health benefits. In some particular aspects, a wearable device for providing vibrotactile stimulus to at least one nerve proximate an ear of a user includes: an earpiece having a vibration device for application of vibrotactile stimulus proximate at least one of a concha region or an ear canal of the user; and a controller connected with the vibration device, the controller configured to actuate the vibration device according to a modulated pattern to stimulate at least one nerve proximate the ear of the user, where the modulated pattern is characterized by: a peak drive level sufficient to stimulate nerve endings of at least one nerve proximate the ear of the user; and a carrier frequency of approximately 50 Hertz (Hz) to approximately 500 Hz.

### PRIORITY CLAIM

This application claims priority to U.S. Provisional Patent Application No. 63/122,604, filed on Dec. 8, 2020, which is incorporated by reference in its entirety.

### TECHNICAL FIELD

This disclosure generally relates to vibrotactile and/or thermal stimulation. More particularly, the disclosure relates to vibrotactile and/or thermal stimulation of nerves proximate the ear, e.g., providing one or more health benefits.

### BACKGROUND

Cranial nerve stimulation (e.g., vagus nerve stimulation (VNS), trigeminal nerve stimulation (TNS), etc.) is used as a treatment for a number of health conditions, e.g., to treat depression, pain, insomnia and/or tinnitus. Many conventional approaches for nerve stimulation rely on electrical stimulation of the auricular branches of cranial nerves. However, electrical nerve stimulation is not always practical or effective.

### SUMMARY

All examples and features mentioned below can be combined in any technically possible way.

Various aspects include wearable devices and related approaches for stimulating nerves in a user, e.g., proximate the user's ear. In certain cases, the nerve stimulation provides one or more health benefits.

In some particular aspects, a wearable device for providing vibrotactile stimulus to at least one nerve proximate an ear of a user includes: an earpiece having a vibration device for application of vibrotactile stimulus proximate at least one of a concha region or an ear canal of the user; and a controller connected with the vibration device, the controller configured to actuate the vibration device according to a modulated pattern to stimulate at least one nerve proximate the ear of the user, where the modulated pattern is characterized by: a peak drive level sufficient to stimulate nerve endings of at least one nerve proximate the ear of the user; and a carrier frequency of approximately 50 Hertz (Hz) to approximately 500 Hz.

In some particular aspects, a wearable device for providing vibrotactile stimulus to at least one nerve proximate an ear of a user includes: a set of two earpieces each having a vibration device for application of vibrotactile stimulus proximate at least one of a concha region or an ear canal of the user's ears; and a controller connected with the vibration devices, the controller configured to actuate the vibration devices according to a modulated pattern to stimulate at least one nerve proximate the ear of the user, where the modulated pattern is characterized by: a peak drive level sufficient to stimulate nerve endings of the at least one nerve proximate the ear of the user; a carrier frequency of approximately 50 Hertz (Hz) to approximately 500 Hz; and a phase difference between the modulated pattern at the vibration devices, where the peak drive level applies a force to at least one of the concha region or the ear canal of the user of approximately 0.3 N to approximately 0.7 N.

In additional particular aspects, a wearable device for providing thermal stimulus to at least one nerve proximate an ear of a user includes: an earpiece having a thermal actuator for application of thermal stimulus proximate at least one of a concha region or an ear canal of the user; and a controller connected with the thermal actuator, the controller configured to actuate the thermal actuator according to a modulated pattern to stimulate at least one nerve proximate the ear of the user, wherein the modulated pattern is characterized by: a peak thermal differential sufficient to stimulate nerve endings of the at least one nerve proximate the ear of the user; and a frequency of 0 Hz (i.e., constant heat) to approximately 1 Hertz (Hz). In certain cases, the frequency is equal to approximately 0.1 Hz to approximately 1 Hz.

Implementations may include one of the following features, or any combination thereof.

In certain implementations, the nerve proximate the user's ear that is stimulated includes at least one of: the vagus nerve, the trigeminal nerve or the facial nerve.

In some cases, the carrier frequency is the fundamental frequency of the vibration device, and is approximately 200 Hz.

In particular aspects, the peak drive level applies a force to at least one of the concha region or the ear canal of the user of approximately 0.1 Newton (N) to approximately 0.9 N.

In certain implementations, the peak drive level applies a force to at least one of the concha region or the ear canal of the user of approximately 0.3 N to approximately 0.7 N.

In some cases, the peak drive level applies a force to at least one of the concha region or the ear canal of the user of approximately 0.5 N.

In certain aspects, the controller is configured to adjust the modulation pattern in response to receiving biometric feedback about the user from a sensor system.

In some implementations, the biometric feedback includes at least one of: heart rate or breath rate, and is detected by a sensor system (e.g., in a connected smart device).

In particular cases, the biometric feedback indicates that the user is exercising or stressed, and the controller adjusts the modulation pattern to an exercise supplemental pattern, an exercise cool down pattern, or a stress reduction pattern.

In certain aspects, the vibration device vibrates at the carrier frequency in response to a stimulation waveform, where the modulated pattern is further characterized by one of: a) a fast onset and a gradual decay, b) a fast onset, a gradual decay, and a period of nominal vibrational force, c) a fast onset, a gradual decay, and a period of zero vibrational force, d) a symmetric onset and decay, or e) a multi-frequency pattern having a first segment with a first frequency and a first period, and a second segment with a second, higher frequency and a second, longer period.

In particular implementations, a nominal vibrational force between pulses (e.g., in pattern (b)) can mitigate jarring as compared with an on/off vibrational pattern.

In certain aspects, the modulated pattern approximates a breath rate of the user and has a period equal to approximately one second to approximately 20 seconds.

In some cases, the period is equal to approximately 7 seconds.

In particular aspects, the wearable device further includes an electro-acoustic transducer connected with the controller and configured to provide an audio output.

In particular implementations, the audio output includes: an audio noise signal configured to entrain the user's breathing, or a noise signal (e.g., white noise or pink noise). In some cases, the audio noise signal includes one or more tone bursts, chirps, or other audio signal(s).

In certain cases, the wearable device further includes a thermal actuator connected with the controller for thermally stimulating an area proximate the ear of the user.

In some implementations, the wearable device includes an in-ear audio device, an on-ear audio device or an over-ear audio device.

In particular aspects, in the case that the wearable audio device includes an on-ear audio device or an over-ear audio device, the earpiece further includes an extension member for positioning proximate at least one of the concha region or the ear canal of the user when the earpiece is positioned on or over the ear of the user.

In certain cases, the wearable device further includes an additional earpiece having an additional vibration device for application of vibrotactile stimulus proximate at least one of a concha region or an ear canal of an additional ear of the user, where the controller is connected with the additional vibration device and is configured to actuate the vibration device and the additional vibration device according to the modulated pattern.

In some cases, the modulated pattern is further characterized by a phase difference between the user's ears.

In certain cases, the phase of the modulated pattern is the same at both ears (zero phase difference).

In particular implementations, the phase difference is equal to 180 degrees.

In some aspects, the phase difference is less than 180 degrees.

In certain cases, the earpiece includes an eartip and a support structure extending from the eartip.

In some implementations, the vibration device is located in the support structure.

In particular aspects, the support structure is formed of a compliant material (e.g., silicone, polyurethane, polynorbornene, thermoplastic elastomer (TPE), and/or fluoroelastomer).

In certain implementations, the vibration device is encased within the support structure or another region of the eartip, e.g., in a hard shell located within the compliant material.

In some aspects, the vibration device includes at least one of: an eccentric rotating mass (ERM) motor, a linear resonant actuator (LRA), a piezoelectric motor, a haptic engine, a focused ultrasound device, or a transducer without a voice coil, and the earpiece is in electrically non-conducting contact with the user's skin during use.

In particular cases, the controller includes a control circuit contained in the earpiece.

In certain implementations, the control circuit is configured to receive commands from at least one of a local logic engine or a remote logic engine for actuating the vibration device according to the modulated pattern.

In some cases, the modulated pattern includes a session of recurring modulated patterns lasting approximately 15-20 minutes or less.

In particular aspects, the session is repeated at least two times throughout a day.

In certain cases, the wearable device further includes a tactile actuator coupled with the controller for providing a low-frequency tactile stimulus to the at least one nerve proximate the ear of the user, where the low-frequency tactile stimulus is characterized by a frequency of approximately ½ Hertz (Hz) or less.

In some aspects, the wearable device further includes an additional vibration device in the earpiece and coupled with the controller, where the controller is configured to actuate the additional vibration device according to the modulated pattern.

In certain implementations, each earpiece further includes an electro-acoustic transducer connected with the controller and configured to provide an audio output, where the audio output includes: an audio signal configured to entrain the user's breathing, or a noise signal.

In some cases, at least one of the earpieces further includes a thermal actuator connected with the controller for thermally stimulating an area proximate at least one of the user's ears.

In particular aspects, in the case that the wearable audio device includes an on-ear audio device or an over-ear audio device, each earpiece further includes an extension member for positioning proximate at least one of the concha region or the ear canal of the user when the earpiece is positioned on or over the ears of the user.

In certain implementations, at least one of the earpieces further includes a tactile actuator connected with the controller for providing a low-frequency tactile stimulus to the at least one nerve proximate the ear of the user, where the low-frequency tactile stimulus is characterized by a frequency of approximately ½ Hertz (Hz) or less.

In some aspects, the peak thermal differential is equal to approximately 0.5 degrees Celsius (C) to approximately 15 degrees C. In certain cases, the peak thermal differential is equal to approximately +15 degrees C. from the user's body temperature to approximately −37 degrees C. from the user's body temperature.

In certain aspects, a frequency of 0 Hz represents application of constant thermal stimulus during the modulated pattern.

Two or more features described in this disclosure, including those described in this summary section, may be combined to form implementations not specifically described herein.

The details of one or more implementations are set forth in the accompanying drawings and the description below. Other features, objects and advantages will be apparent from the description and drawings, and from the claims.

### DESCRIPTION OF THE DRAWINGS

FIG. 1A is a front view of an example earpiece capable of nerve stimulation according to various implementations.

FIG. 1B is a rear view of the example earpiece of FIG. 1A.

FIG. 1C is a side view of the earpiece of FIG. 1A.

FIG. 1D is an exploded front view of the earpiece of FIG. 1A.

FIG. 1E is an exploded front view of the earpiece of FIG. 1A.

FIG. 2 is a schematic depiction of a nerve stimulation system including the earpiece of FIG. 1A, according to various implementations.

FIG. 3 is a schematic view of an electronics module from the nerve stimulation system of FIG. 2, according to various implementations.

FIG. 4 is an example view of another earpiece capable of nerve stimulation, according to various implementations.

FIG. 5 is a view of the lateral surface of the human ear.

FIG. 6A is an exploded perspective view of another earpiece capable of nerve stimulation, according to various implementations.

FIG. 6B is a rear view of the earpiece of FIG. 6A.

FIG. 7A is a front view of another earpiece capable of nerve stimulation, according to various implementations.

FIG. 7B is a side view of the earpiece of FIG. 7A.

FIG. 7C is an exploded front view of the earpiece of FIG. 7A.

FIGS. 7D and 7E are end views of the earpiece of FIG. 7A, illustrating quadrupole motion of an ear tip.

FIG. 8 is a cross-sectional side view of another earpiece capable of nerve stimulation, according to various implementations.

FIG. 9 is a cross-sectional view of another earpiece capable of nerve stimulation, according to various implementations.

FIGS. 10A through 10G are perspective, front, rear, left side, right side, top, and bottom views, respectively, of an open audio device designed for the right ear, according to various implementations.

FIG. 11 is a side view of the open audio device of FIGS. 10A-10G mounted on a user's right ear.

FIGS. 12A-12E are perspective, rear, left side, right side, and top views, respectively, of an open audio device with a protrusion for applying vibrotactile stimulation to a user's cyma concha and designed for the right ear, according to various implementations.

FIG. 13 illustrates an additional earpiece according to various implementations.

FIG. 14 illustrates an earpiece according to various further implementations.

FIG. 15 illustrates an earpiece according to still further implementations.

FIG. 16 illustrates an additional earpiece according to various implementations.

FIG. 17 illustrates a further implementation of an earpiece.

FIG. 18 shows a set of single ear waveform graphs for applying a modulated nerve stimulation pattern according to various implementations.

FIG. 19 shows a set of bilateral waveform graphs for applying a modulated nerve stimulation pattern according to various implementations.

It is noted that the drawings of the various implementations are not necessarily to scale. The drawings are intended to depict only typical aspects of the disclosure, and therefore should not be considered as limiting the scope of the invention. In the drawings, like numbering represents like elements between the drawings.

### DETAILED DESCRIPTION

As noted herein, various aspects of the disclosure generally relate to cranial nerve stimulation. More particularly, aspects of the disclosure relate to vibrotactile and/or thermal stimulation of one or more cranial nerves such as nerves proximate a user's ear.

Commonly labeled components in the FIGURES are considered to be substantially equivalent components for the purposes of illustration, and redundant discussion of those components is omitted for clarity.

Aspects and implementations disclosed herein may be applicable to a wide variety of wearable devices, such as wearable audio devices in various form factors, e.g., headphones (whether on or off ear), headsets, eyeglasses, audio sleeping aids, etc. Some particular aspects disclosed may be particularly applicable to personal (wearable) audio devices such as in-ear headphones (also referred to as earbuds), eyeglasses or other head-mounted audio devices.

While described by way of example, wearable (audio) devices such as in-ear headphones (e.g., earbuds), over-ear headphones, or audio eyeglasses (also referred to as eyeglass headphones) herein, the wearable (e.g., audio) devices disclosed herein can include additional features and capabilities. That is, the wearable devices described according to various implementations can include features found in one or more other wearable electronic devices, such as smart glasses, smart watches, etc. These wearable devices can include additional hardware components, such as one or more cameras, location tracking devices, microphones, etc., and may be capable of voice recognition, visual recognition, and other smart device functions. The description of wearable devices included herein is not intended to exclude these additional capabilities in such a device.

Various implementations include wearable devices (e.g., wearable electronic devices and/or wearable audio devices) and related approaches for providing vibrotactile and/or thermal stimulus to at least one nerve proximate a user's ear. In contrast to conventional approaches, the devices and approaches described according to various implementations provide non-electrical stimulation of one or more neural pathways proximate the ear in order to provide one or more health benefits. In certain implementations, a wearable device is configured to stimulate one or more nerves proximate the user's ear with a vibration device that is actuated according to a modulated pattern. In additional implementations, a wearable device is configured to stimulate one or more nerves proximate the user's ear with a thermal actuator that is actuated according to a modulated pattern.

Certain approaches for providing vibrotactile stimulation of the auricular branch of the vagus nerve are described in U.S. Provisional Patent Application No. 63/044,078 filed on Jun. 25, 2020 (“Vibrotactile Stimulation,” Attorney Docket No. WL-20-080-US), which is incorporated by reference in its entirety. Additionally, certain approaches for controlling stimulation of nerves are described in U.S. Provisional Patent Application No. 63/042,606 (“Haptic Tinnitus Therapeutic System”, filed on Jun. 23, 2020), which is incorporated by reference in its entirety.

Example Wearable Devices

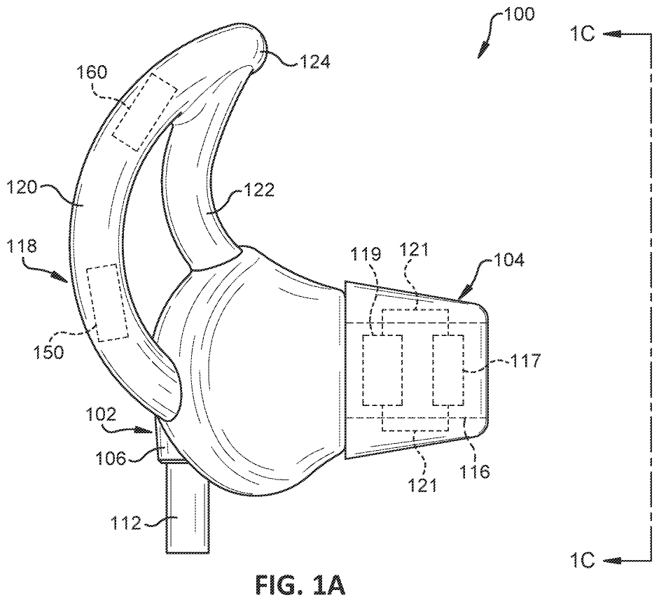

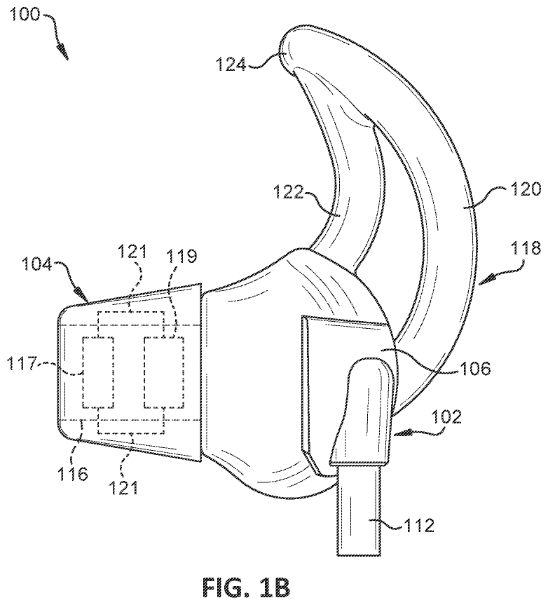

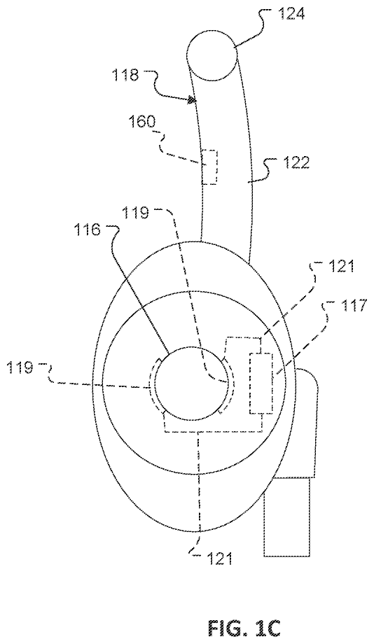

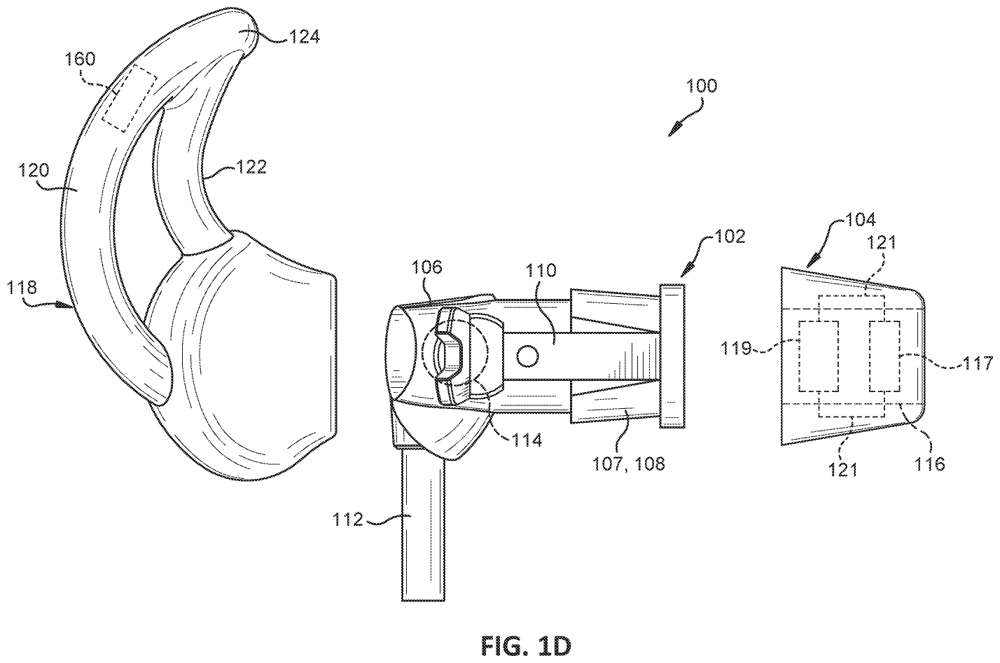

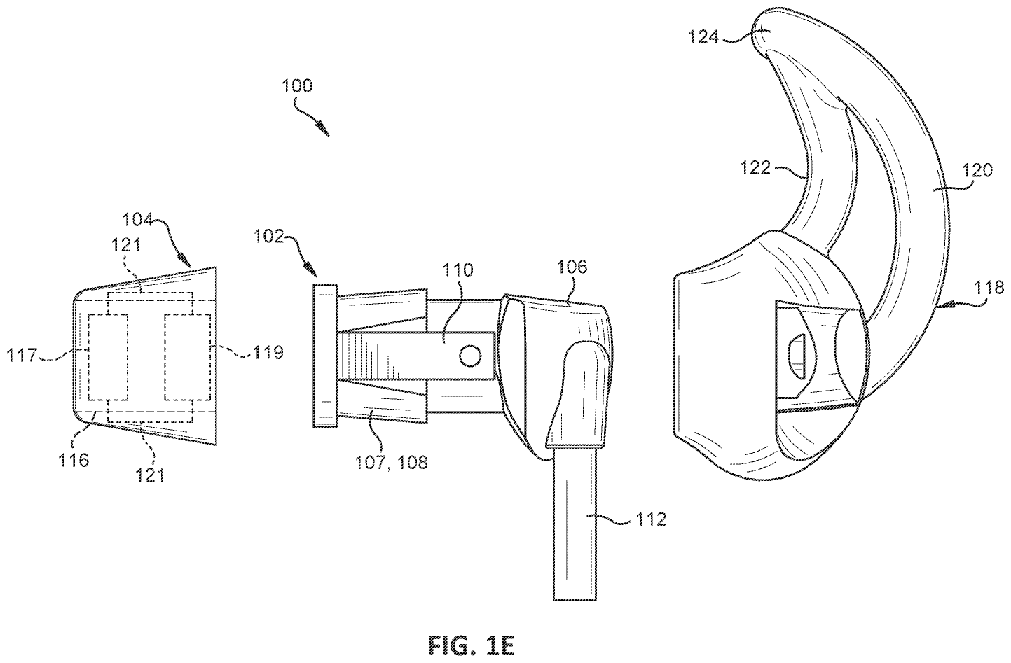

With reference to FIGS. 1A through 1E, an example earpiece 100 in a wearable device is shown for delivering nerve stimulation proximate a user's ear. In various implementations, the wearable device includes two earpieces such as the earpiece 100 shown in FIGS. 1A through 1E. However, in other cases, the wearable device includes only a single such earpiece. As further described herein, wearable devices in other form factors can employ various implementations of the disclosure, and as such, the earpiece 100 shown in FIGS. 1A through 1E is not intended to limit these implementations.

Returning to FIGS. 1A through 1E, the example earpiece 100 includes an earbud 102 and an ear tip 104. The earbud 102 includes a rigid housing 106 that defines a protrusion 107 in the form of a nozzle 108 which supports a pair of electrodes 110. The housing 106 may be formed of, e.g., molded from, a hard plastic such as Acrylonitrile Butadiene Styrene (ABS), Polycarbonate/Acrylonitrile Butadiene Styrene (PCB/ABS), polyetherimide (PEI), or stereolithography (SLA) resin. In certain implementations, wiring 112 extends into the housing 106 and couples to the electrodes 110 for providing an electrical current to the electrodes 110. In some cases, e.g., when the wearable device includes a wearable audio device, the earpiece 100 includes an electro-acoustic transducer 114 (FIG. 1D) supported in the housing 106. The electro-acoustic transducer 114 may be acoustically coupled to an acoustic passage in the nozzle 108 such that the electro-acoustic transducer 114 can be acoustically coupled to a user's ear canal when the earpiece is worn. The electro-acoustic transducer 114 can be used to deliver audio content, e.g., entertainment audio, such as music, or therapeutic audio such as guided meditation or guided breathing. In some cases, a nerve stimulation treatment, applied via the electrodes 110, is coordinated with guided breathing audio, such as described in U.S. patent application Ser. No. 16/567,116, titled “SYSTEMS AND METHODS FOR PROVIDING AND COORDINATING VAGUS NERVE STIMULATION WITH AUDIO THERAPY,” (attorney docket no. 2115P064/WL-19-060-US) filed Sep. 11, 2019 and incorporated herein by reference. In some cases, the audio may be white noise with a notch at the user's tinnitus percept frequency; i.e., a notch at the frequency at which the user perceives tinnitus. In other cases, the audio includes pink noise.

In this example, the ear tip 104 is in the shape of hollow cylinder with a hollow passage 116 that is configured to receive the nozzle 108 of the earbud 102 such that the ear tip 104 overlies the electrodes 110. Notably, the ear tip 104 may support a vibration device 117 in various implementations. The ear tip 104 is configured to be received within a user's ear canal and such that the vibration device 117 is at least partially disposed within the ear canal. The ear tip 104 may be formed of a compliant material that can conform to the user's ear geometry to help ensure a tight fit, and good, distributed contact with the user's ear canal. Suitable materials for the ear tip 104 include soft flexible materials such as silicone, polyurethane, polynorbornene (e.g., Norsorex® material available from D-NOV GmbH of Vienna, Austria), thermoplastic elastomer (TPE), and/or fluoroelastomer.

The vibration device 117 may be housed or suspended in the material that forms the ear tip 104. In some cases, the ear tip 104 may be formed around the vibration device 117 in an insert molding process. In other implementations, the vibration device 117 is encased within the material of the ear tip 104, e.g., in a shell that is partially compliant, or includes a stiff core with a compliant outer layer. The vibration device 117 may be arranged adjacent the hollow passage 116. In certain cases, the vibration device 117 may be electrically connected to electrical contact pads 119 arranged along an inner surface of the hollow passage 116. The electrical contact pads 119 are arranged to overlie and electrically couple to the electrodes 110 on the earbud 102 for delivering power to the vibration device 117. The vibration device 117 can be electrically connected to the electrical contact pads 119 via electrical traces 121 (e.g., metal wires or conductive leads suspended in the material forming the ear tip 104.

In certain implementations, the vibration device 117 is arranged to deliver vibrotactile stimulation to a user, e.g., to the auricular branch of the user's vagus nerve, when worn. In that regard, the vibration device 117 may be arranged to deliver stimulation to dorsal and/or ventral surfaces of the ear canal. In various additional implementations, one or more vibration devices 117 are positioned to apply vibrotactile stimulus proximate at least one of the concha region or the ear canal of the user. The vibration device 117 may include at least one of: an eccentric rotating mass (ERM) motor, a linear resonant actuator (LRA) a piezoelectric motor, a haptic motor engine such as the “taptic engine” found in various Apple products, a focused ultrasound device, or a transducer without a cone. In various particular implementations, as noted herein, the earpiece 100 is configured to vibrate to stimulate one or more nerves proximate the user's ear while remaining in electrically non-conducting contact with the user's skin.

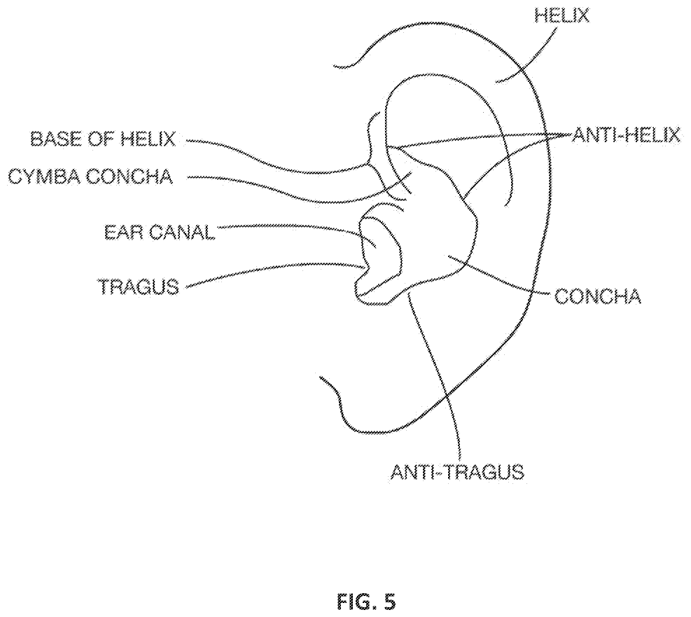

As shown in FIGS. 1A through 1E, the earpiece 100 may also include a support structure (also called a “positioning and retaining structure”) 118. In implementations where the earpiece 100 is an in-ear wearable device (e.g., an in-ear audio device such as an earbud), the support structure 118 can help to keep the earpiece 100 seated in the user's ear. As shown in the illustrated example, the support structure 118 may be formed separately from the ear tip 104 and is configured to be coupled (e.g., releasably coupled) to the earbud 102. In certain cases, the support structure 118 includes an outer leg 120 and an inner leg 122. The outer leg 120 is curved to generally follow the curve of the anti-helix (FIG. 5) at the rear of the concha of the subject's ear. Distal ends of the legs 120, 122 are joined at a point 124. The support structure 118 may be formed of, e.g., molded from a compliant material such as silicone, polyurethane, thermoplastic elastomer (TPE), and/or fluoroelastomer.

Although FIGS. 1A through 1E show retaining legs 120, 122 as one embodiment of the support structure 118, this disclosure is not limited to such a configuration. Any type of support structure is contemplated. For example, in some cases, the support structure may include only a single leg. In other cases, the distal end of the inner leg may be joined to the outer leg at some point other than at the distal end of the outer leg. Alternatively, the support structure can be omitted altogether.

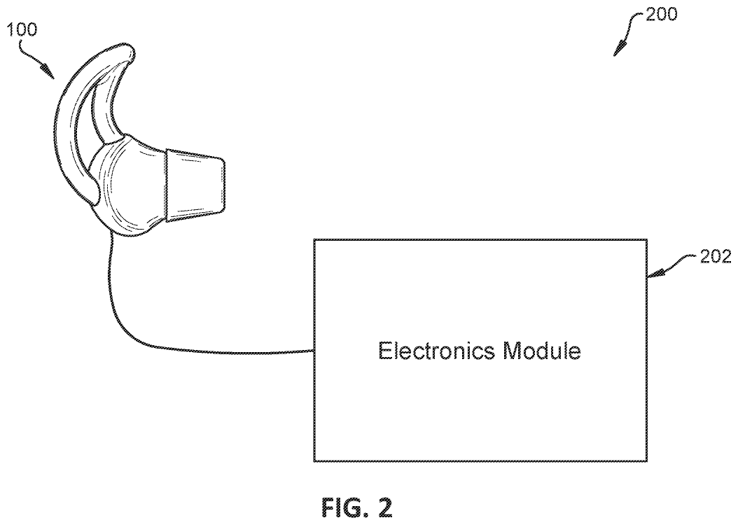

As shown in FIG. 2, the earpiece 100 may be incorporated into a nerve stimulation (NS) system 200. The system 200 includes the earpiece(s) 100 and an electronics module 202, which is coupled to the earpiece(s) 100 via the wiring 112. In other cases, the electronics module 202 is wirelessly coupled with the earpiece(s) 100, e.g., via one or more wireless transceivers. The electronics module 202 houses the electronics for powering the earpiece 100. The electronics module 202 includes the programming for providing a current waveform as described herein, to the vibration device 117, e.g., via the electrodes 110, for nerve stimulation treatment. The electronics module 202 may also include a user interface, e.g., hardware buttons or a graphical user interface, to all the user or a clinician to adjust settings.

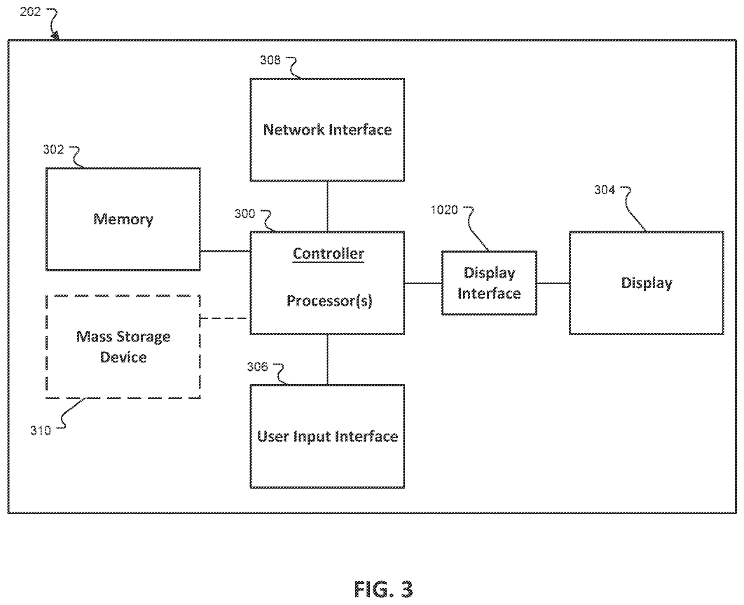

Referring to FIG. 3, an example electronics module 202 includes a controller (e.g., including a processor with a logic engine) 300, a memory 302, a display 304, a user input interface 306, and a network interface 308, among other components. The electronics module 202 may also be provided with a mass storage device 310, such as a hard drive, a flash drive, or other device, to provide additional storage. Each of the controller 300, the memory 302, the display 304, and the network interface 308 are interconnected using various buses, and several of the components may be mounted on a common motherboard or in other manners as appropriate.

In certain cases, the controller 300 includes a control circuit coupled with the processor and/or logic engine for actuating vibration of the vibration device(s) 117 according to a prescribed pattern. In some particular cases, a control circuit is contained in one or both earpieces 100, and receives commands from the logic engine for actuating vibration of the vibration device(s) 117 according to a prescribed pattern. In additional cases, a logic engine is located remotely relative to the earpiece 100, e.g., in a connected smart devices such as a smart phone, smart watch, wearable smart device, etc., or in a cloud-based logic engine that is accessible via communications components at the electronics module 202 (e.g., network interface 308).

The controller (including processor) 300 can execute instructions (e.g., software) within the electronics module 202, including instructions stored in the memory 302 or in a secondary storage device (e.g., mass storage device 310). The controller 300 may be implemented as a chipset of chips that include separate and multiple analog and digital processors. The controller 300 may provide, for example, for coordination of other components of the electronics module 202, such as control of user interfaces, applications run by the electronics module 202, and network communication by the electronics module 202. The controller 300 may manage communication with a user through the display 304 and the user input interface 306.

The controller 300 may communicate with the user through a display interface 312 coupled to the display 304. The display 304 may include an LCD monitor, or a touch sensitive display (e.g., in the case of a mobile device). The display interface 312 may comprise appropriate circuitry for driving the display 304 to preset graphical and other information to the user. The user input interface 306 may include one or more user input devices such as a keyboard, a pointer device such as a mouse, and/or a touch sensitive display. In some cases, the same device (e.g., a touch sensitive display) may be utilized to provide the functions of the display 304 and the user input interface 306. The user input interface 306 may be used, for example, to receive input from the user that can be used to identify a symptom of a particular health condition (e.g., a tinnitus percept, a perceived stress level, a desired breath rate) experienced by the user.

The memory 302 stores information within the electronics module 202. In some implementations, the memory 302 is a volatile memory unit or units. In some implementations, the memory 302 is a non-volatile memory unit or units. The memory 302 may also be another form of computer-readable medium, such as magnetic or optical disk. The mass storage device 310 can provide mass storage for the electronics module 202. In some implementations, the mass storage device 310 may be or contain a computer readable medium, such as a floppy disk device, a hard disk device, an optical disk device, or a tape device, a flash memory or other similar solid-state memory device, or an array of devices. Instructions (e.g., software) can be stored in an information carrier. The instructions, when executed by one or more processing devices (e.g., the processor 300), perform one or more processes, such as generating current waveforms for nerve stimulation therapy and/or generating audio. In some particular cases, the generated audio may include an audio signal that consists of broadband noise with a notch at the frequency (the percept frequency) at which the user perceives tinnitus. The audio may also include test tones that are used to diagnose/identify the user's tinnitus percept frequency. The instructions can also be stored by one or more storage devices such as computer- or machine-readable mediums (for example, the memory 302, the storage device 310, or memory in the processor 300).

In some cases, one or more components of the electronics module may be housed within the earbud or ear tip.

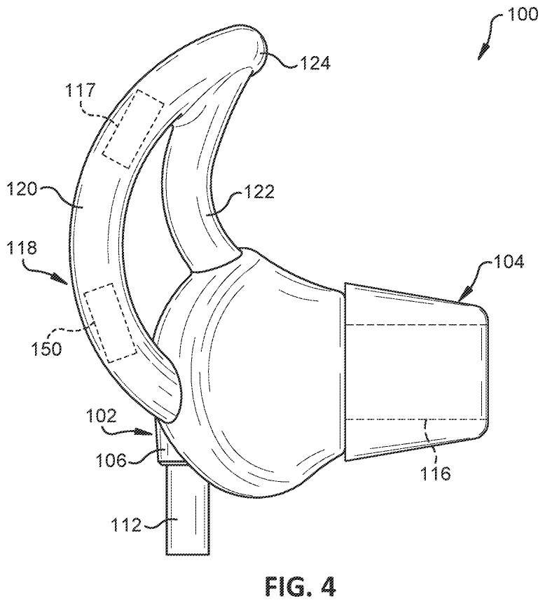

FIG. 4 illustrates a further configuration of the earpiece 100 in which the vibration device 117 is arranged in the support structure 118. In these implementations, the vibration device 117 is arranged to provide vibrotactile stimulation to a cymba concha (FIG. 5) of a user's ear. Similarly to the description of the vibration device with respect to FIGS. 1A through 1E, the retaining structure 118 may be formed, e.g., molded, around the vibration device 117, or the vibration device 117 can be encased within the retaining structure 118. In cases where the vibration device 117 is controlled or otherwise powered via a wired connection, wiring may be run through the outer and/or inner legs 120, 122 and may terminate at electrical contact pads on an inner surface of the support structure 118 which may overlie and establish electrical contact with electrodes on the surface of the earbud 102. As noted herein, the vibration device 117 can include one or more of: an ERM motor, a LRA, a piezoelectric motor, a haptic engine, a focused ultrasound device or a transducer without a voice coil.

FIG. 5 shows an external portion of a human (left) ear, also known as the outer ear, with some features identified. The outer ear is the portion of the ear that can be seen by casual inspection. It consists of the pinna (what we generally call the ‘ear’), which is attached to a bowl-shaped structure called the concha. The concha ends at the ear canal. There are many different ear sizes and geometries. Some ears have additional features that are not shown in FIG. 5. Some ears lack some of the features that are shown in FIG. 5. Some features may be more or less prominent than are shown in FIG. 5.

While implementations have been shown and described in which the ear tip 104 and the support structure 118 are separately formed pieces, in some implementations, the ear tip and the positioning and retaining structure may alternatively be integrally formed, e.g., in a single molding operation. Still, in other implementations, the vibration device(s) may be disposed in other locations, such as in the earbud. For example, the vibration device may be arranged to rest at least partially within a user's concha when worn. In some cases, the vibration device 117 is arranged to rest partially within the user's concha and partially within the user's ear canal when worn. In other implementations, the vibration device 117 may be arranged to sit entirely within a user's concha cavum, when worn.

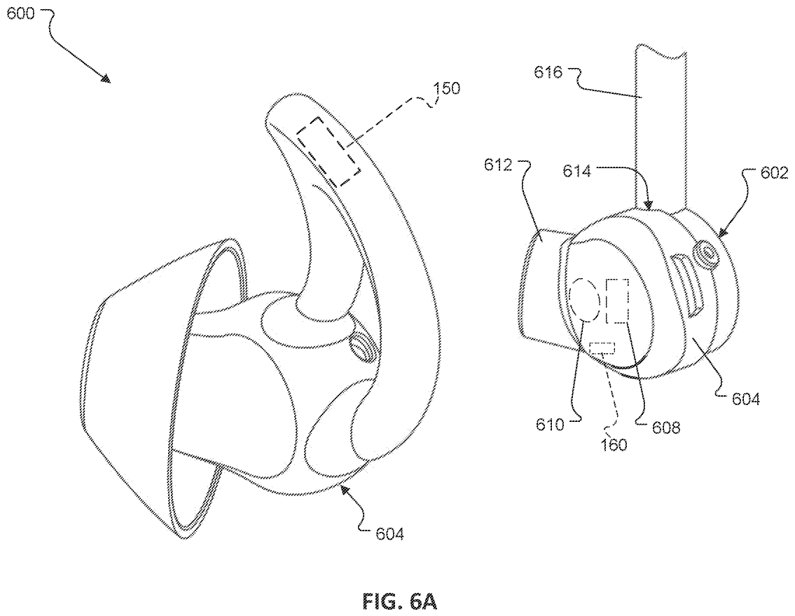

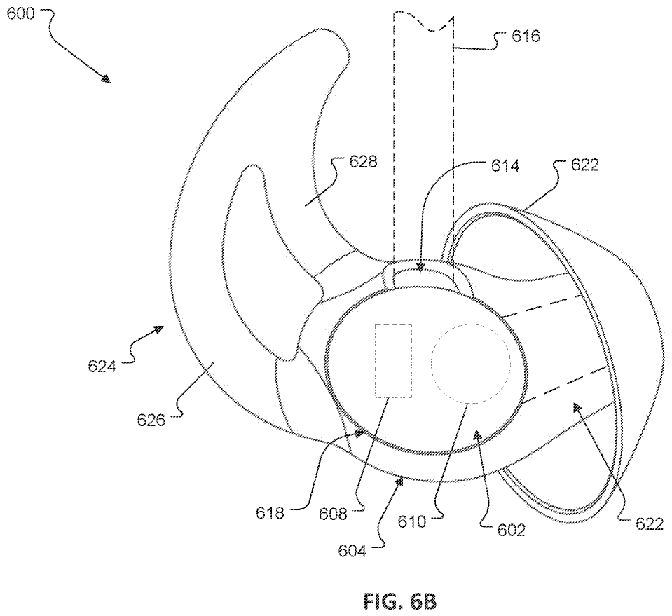

For example, with reference to FIGS. 6A & 6B, another example earpiece 600 for delivering VNS therapy includes an earbud 602 and an ear tip 604. The earbud 602 includes a rigid housing 606 that carries a vibration device 608 and an electro-acoustic transducer 610. In some cases, the vibration device 608 may be supported on or extend through an outer surface of the housing 606, i.e., such that the vibration device 608 directly contacts the ear tip 604. Alternatively, the vibration device 608 may be enclosed within the housing 606, such that the vibrotactile stimulus is transmitted from the motor 608 to the housing 606, and, then, from the housing 606 to and through the ear tip 604 to the user's concha cavum. As noted with respect to the vibration device 117 herein, the vibration device 608 can include one or more of: an ERM motor, a LRA, a piezoelectric motor, a haptic engine, a focused ultrasound device or a transducer without a voice coil.

The housing 606 defines a nozzle 612 (FIG. 6A) that is configured to allow acoustic energy radiated from the transducer 610 to pass therethrough. The housing 606 also defines a wiring receptacle 614 that is configured to receive wiring 616 for powering the vibration device 608 and the transducer 610. The housing 606 may have a two-piece construction and may be formed from a molded plastic such as acrylonitrile butadiene styrene (ABS), polycarbonate (PC), PC/ABS, polyamide (PA) 6,6, or PA 12.

The ear tip 604 is an elastomeric cover that couples to the earbud 602 and provides a relatively soft contact surface for engaging the user's ear. The eartip 604 defines a cavity 618 for receiving the earbud 602 and a hollow passage 620 that surrounds and extends the nozzle 612 (FIG. 6A) formed in the earbud 602. The nozzle 612 and hollow passage 620 together providing an acoustic path for acoustically coupling the transducer 610 to a user's ear canal. The eartip 604 also defines an umbrella-shaped tip 622 that surrounds the hollow passage 620 and is configured to engage a user's ear canal. In the illustrated example, the ear tip 604 also defines a support structure 624 that includes an outer leg 626 and an inner leg 628. The outer leg 626 is curved to generally follow the curve of the anti-helix at the rear of the concha of the user's ear. Although FIGS. 6A and 6B show retaining legs 626, 628 as one embodiment of the support structure 624, this disclosure is not limited to such a configuration. Any type of support structure is contemplated. Alternatively, the support structure 624 can be omitted altogether. The ear tip 604 can be made of any suitable soft, flexible materials, including, for example, silicone, polyurethane, polynorbornene (e.g., Norsorex® material available from D-NOV GmbH of Vienna, Austria), thermoplastic elastomer (TPE), and/or fluoroelastomer.

In some cases, the vibration device may be arranged to be disposed in a user's concha cavum and may be coupled to a portion of the earpiece that sits within the user's ear canal or rests against the user's anti-helix via a mechanical coupling such as a linkage, such that vibrotactile stimulation is transmitted beyond the user's concha cavum; i.e., as an alternative or in addition to providing stimulus to the user's concha cavum.

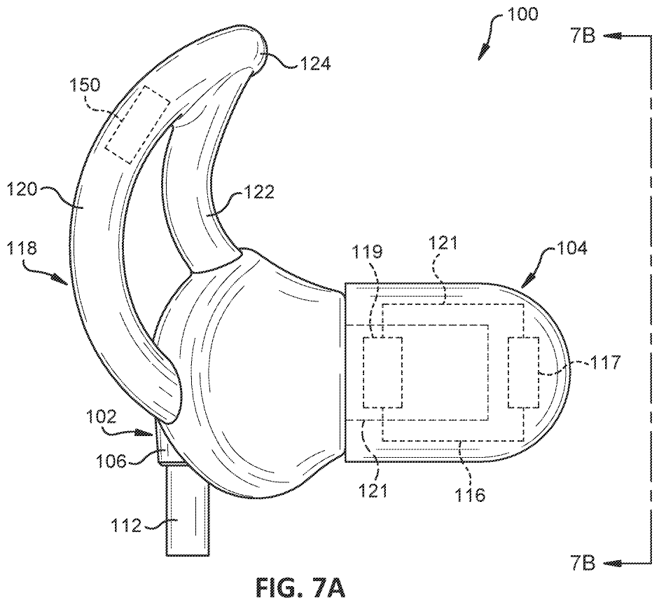

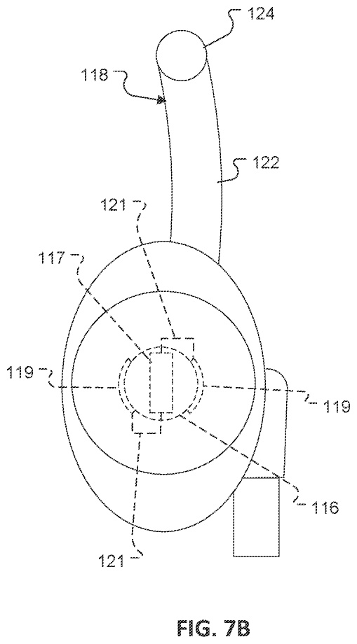

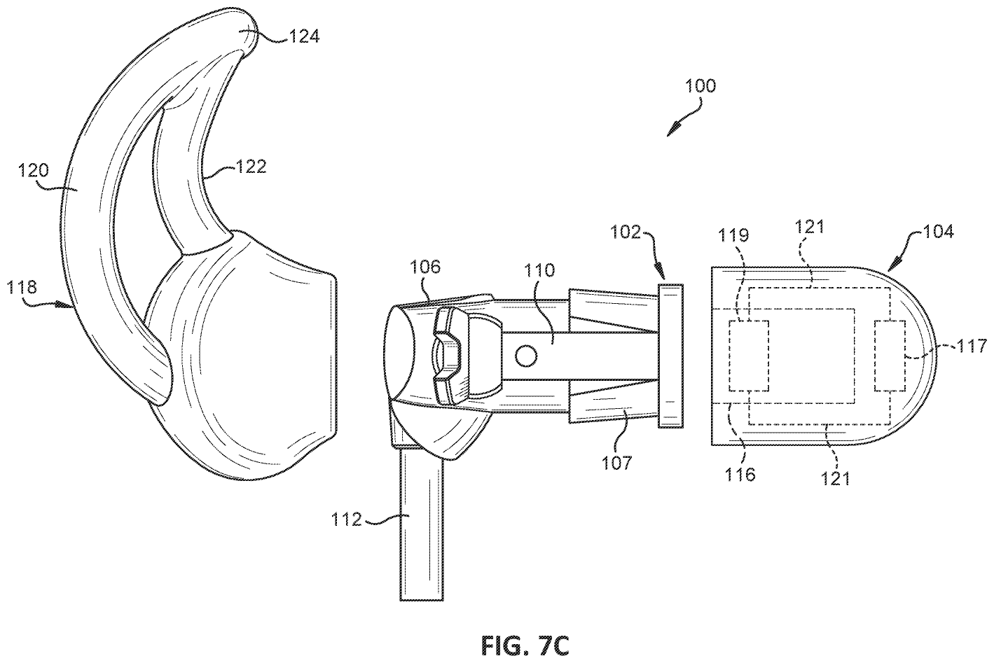

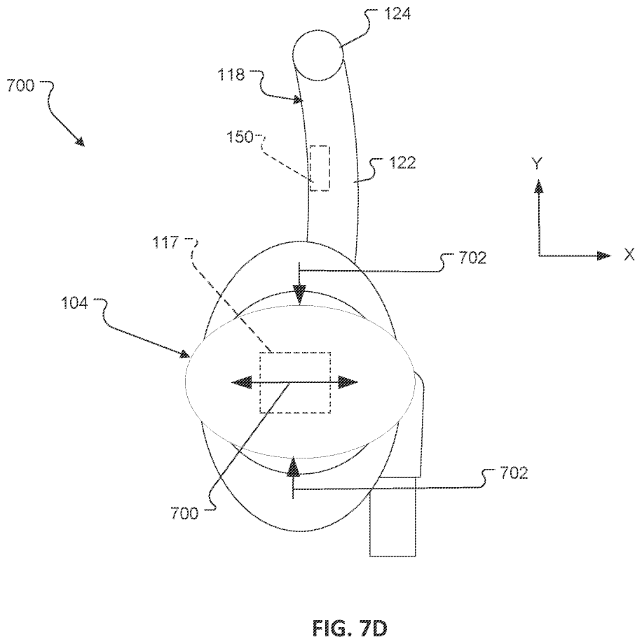

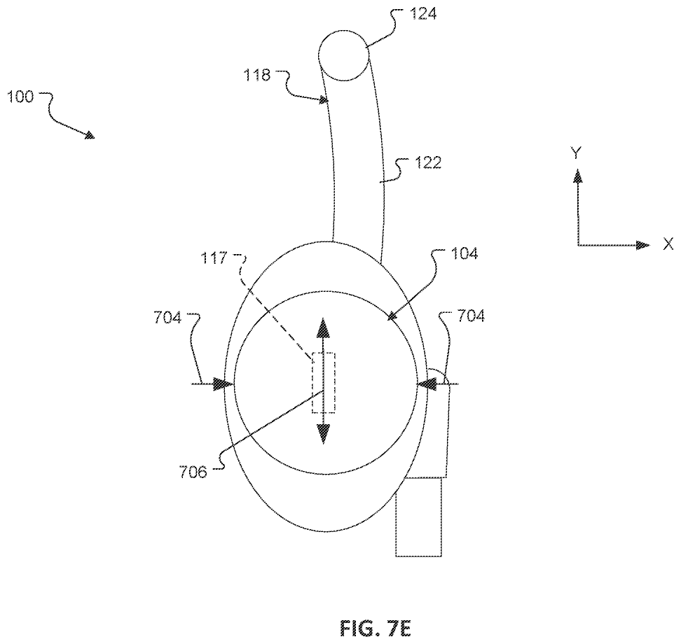

While an implementation has been described in which the earpiece 100 includes an electro-acoustic transducer (item 114, FIG. 1D), some implementations may not include an electro-acoustic transducer. For example, FIGS. 7A-7C illustrate an implementation in which there is no electro-acoustic transducer. Since there is no electro-acoustic transducer, there is no need for a nozzle 108, as a result the vibration device 117 can be arranged coaxially with the protrusion 107 on the earbud 102. In these cases the vibration device 117 is arranged to lie within a user's ear canal when the earpiece 100 is worn. Electrical traces 121 electrically connect the vibration device 117 to electrical contacts 119 arranged on an inner surface(s) of the ear tip 104. The electrical contacts 119 are arranged to overlie and establish an electrical contact with the electrodes 110 supported on the earbud 102. In some cases, the earpiece may consist essentially of just and ear tip with the vibration device, and the vibration device may be connected to the electronics via wiring that runs between the electronics and the ear tip.

In certain implementations, the vibration device 117 may be configured to extend and contract along a linear axis. For example, the vibration device 117 may be a piezoelectric motor that expands and contracts along a linear axis. With reference to FIGS. 7D and 7E, the vibration device 117 is arranged to drive the ear tip 104 in a quadrupole motion in which the ear tip 104 expands (arrows 700, FIG. 7D) along an x-axis and contracts (arrows 702, FIG. 7D) along a y-axis when the vibration device 117 expands, and, in which the ear tip 104 contracts (arrows 704, FIG. 7E) along the x-axis and expands (arrows 706, FIG. 7E) along the y-axis when the vibration device 117 contracts. The ear tip 104 is arranged such that the x-axis extends through the dorsal and ventral surfaces of a user's ear canal when the earpiece 100 is worn, so as to deliver stimulation to dorsal and/or ventral surfaces of the user's ear canal via the movements of the vibration device 117 and ear tip 104. In some cases, the fully contracted position, FIG. 7E, may be the rest position of the vibration device 117.

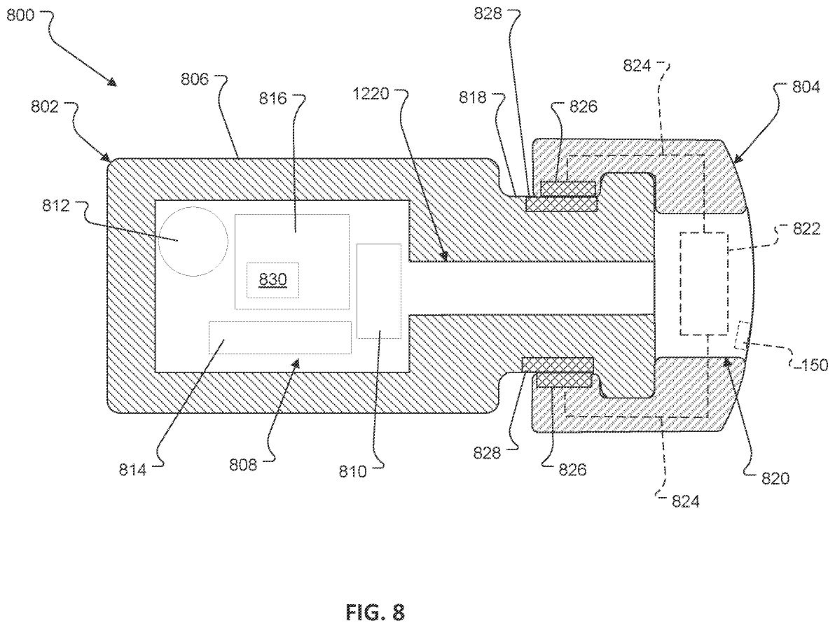

FIG. 8 illustrates another example of an earpiece 800 that includes an earbud 802 and an ear tip 804. In the illustrated example, the earbud 800 includes a housing 806 that defines a cavity 808 within which an electro-acoustic transducer 810, a microphone 812, a battery 814, and a processor 816 are disposed. The cavity 808 is acoustically coupled to an acoustic passage in a nozzle 818 of the earbud 802, e.g., such that the electro-acoustic transducer 810 can be acoustically coupled to a user's ear canal when the earpiece is worn. A hollow passage 820 in the ear tip 804 is coupled to the nozzle 818. The ear tip 804 supports a vibration device 822, e.g., for proving vibrotactile stimulus to the user's vagus nerve. Electrical traces 824, e.g., electrically conductive wires, electrically connect the vibration device 822 to electrical contact pads 826 arranged on an inner surface of the ear tip 804, e.g., along a surface defining the hollow passage 820. The electrical contact pads 826 overlie and establish an electrical connection with electrodes 828 arranged on the surface of the earbud 802, which, in turn, are electrically coupled to the electronics, e.g., the processor 816 in the cavity 808.

The processor 816 may be configured to execute an active noise cancellation (ANC) algorithm 830. In that regard, the microphone 812 is arranged to pick up audio in the user's ear canal. The ANC algorithm 830 uses input from the microphone 812 to provide an anti-noise signal that is used to drive the electro-acoustic transducer 810 to produce an acoustic output to cancel acoustic energy in the user's ear canal that is attributable to the operation of the vibration motor 820.

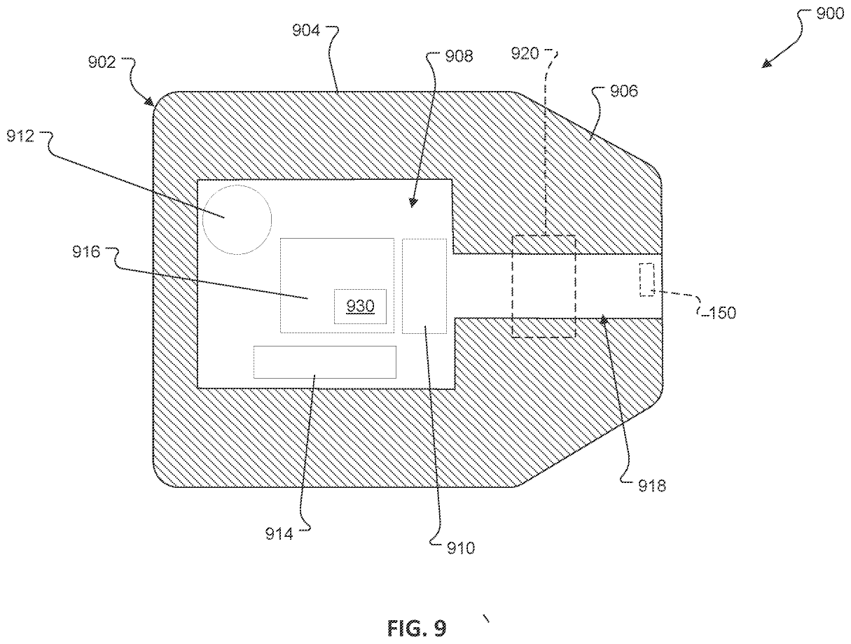

Although implementations have been described in which an earpiece includes an earbud and an ear tip coupled to the earbud, FIG. 9 illustrates another implementation of an earpiece 900 that includes an earbud 902 without an ear tip. The earbud 902 is configured to engage a user's ear canal directly to form an acoustic seal therebetween. The earbud 902 includes a housing 904 that defines a nozzle 906 that is configured to engage a user's ear canal. The housing 904 may be formed of, e.g., molded form, a hard plastic such as those described above. The housing 904 defines a cavity 908 within which an electro-acoustic transducer 910, a microphone 912, a battery 914, and a processor 916. The cavity 908 is acoustically coupled to an acoustic passage 918 in the nozzle 906, e.g., such that the electro-acoustic transducer 910 can be acoustically coupled to a user's ear when the earpiece is worn. Notably, the earbud 902 may support a vibration device 920, which may take a similar form as any vibration device described herein. The vibration device 920 may be arranged adjacent the acoustic passage 918 and is electrically coupled to the electronics, e.g., the processor 816 in the cavity 808, via electrical traces (not shown). The earbud 902 is configured to be received within a user's ear canal and such that the vibration device 920 is at least partially disposed within the ear canal.

As in the implementation discussed above with respect to FIG. 8, the processor 916 of the implementation of FIG. 9 may be configured to execute an active noise cancellation (ANC) algorithm 930. In that regard, the microphone 912 is arranged to pick up audio in the user's ear canal. The ANC algorithm 930 uses input from the microphone 912 to provide an anti-noise signal that is used to drive the electro-acoustic transducer 910 to produce an acoustic output to cancel acoustic energy in the user's ear canal that is attributable to the operation of the vibration motor 920.

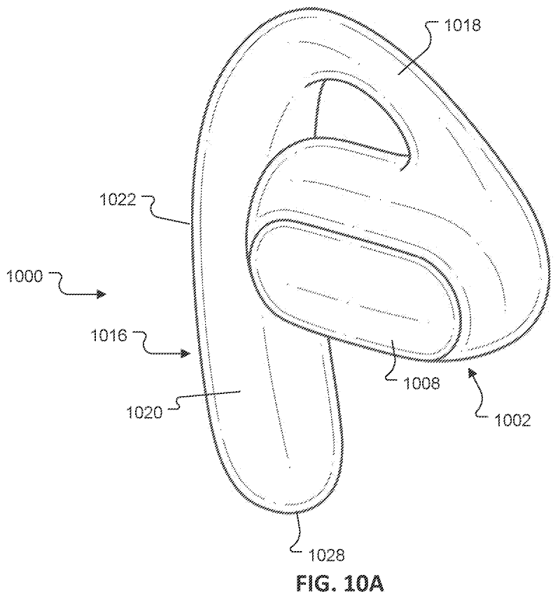

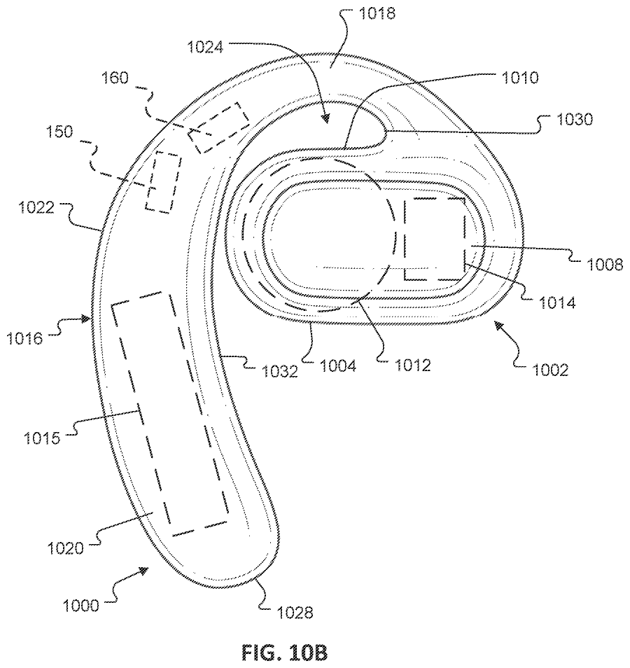

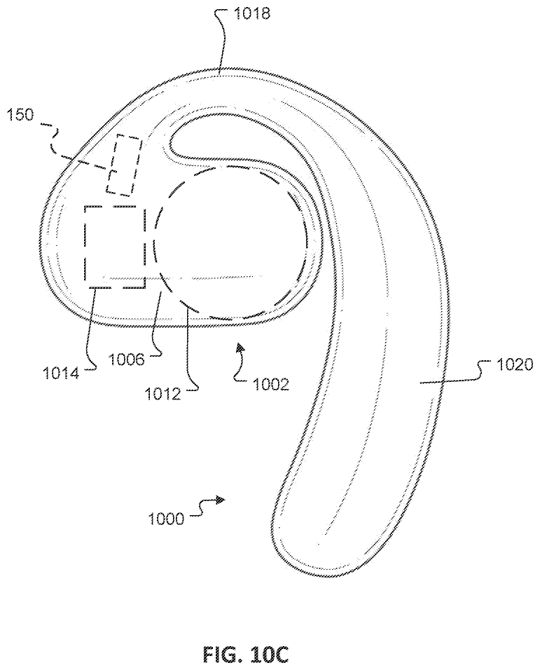

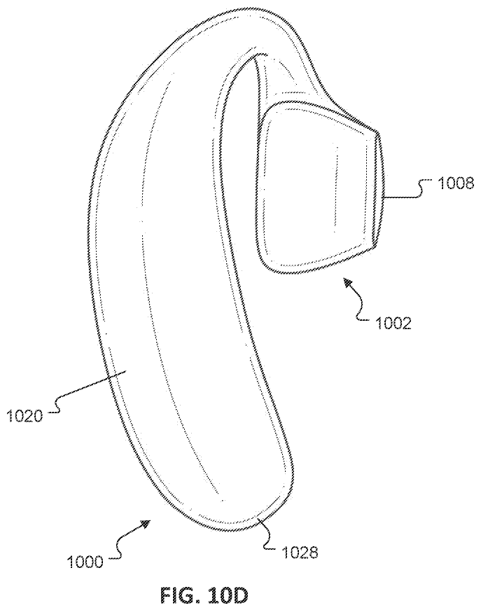

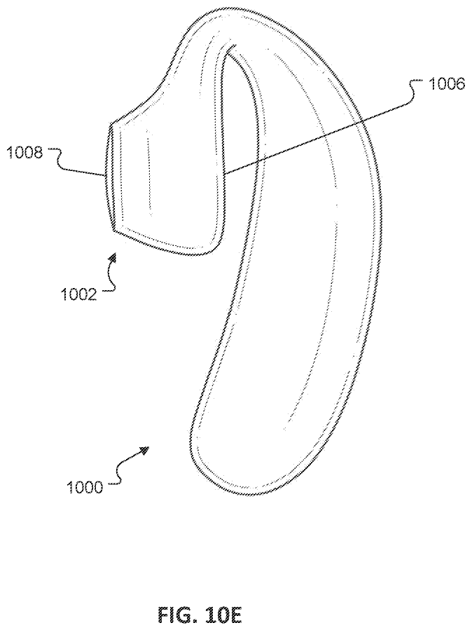

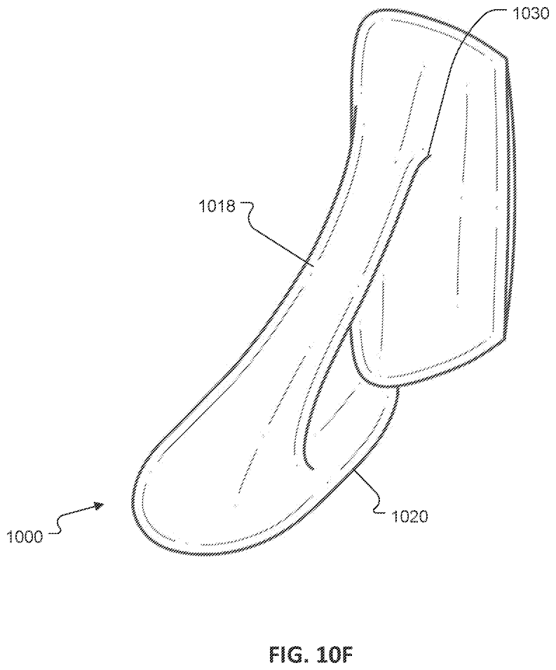

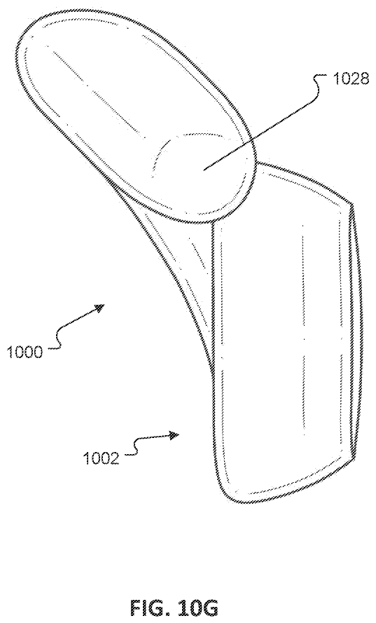

FIGS. 10A-10G illustrate an example of an open audio device 1000 that is configured as an open earphone. The open audio device 1000 illustrated in FIGS. 10A-10G is specifically designed to be carried on the right ear. An open audio device for the left ear would be a mirror image that that shown. The open audio device 1000 is carried by outer ear and portions and of the head that are adjacent to the ear, as is further described elsewhere herein. Open audio device 1000 comprises acoustic module 1002 that is configured to locate sound-emitting opening 1004 above the ear canal opening, which is behind (i.e., generally underneath) ear tragus. Acoustic module 1002 has inner face 1006 and opposed outer face 1008. Beneficially, positioning the acoustic module 1002 above the ear canal opening leaves the ear canal opening unobstructed when viewed from both the side and front, which visually signals to others around the user that the user is open and able to interact with his or her environment. In an example acoustic module 1002 has a second sound-emitting opening 1010 that is farther from the ear canal than opening 1004. Openings 1004 and 1010 can emit sound from opposite sides (front and back) of an electro-acoustic transducer 1012 and so the sounds are out of phase. The out of phase sounds will tend to cancel in the far field and so the openings act like a low-frequency dipole. However, opening 1004 is close enough to the ear canal that its sound is not cancelled before it reaches the ear.

The acoustic module 1002 supports the electro-acoustic transducer 1012 as well as a vibration device 1014. The vibration device 1014 is arranged such that it overlies the user's cymba concha when worn; i.e., such that vibrotactile stimulation from the vibration device 1014 is transmitted to the user's cymba concha. The vibration device 1014 may include at least one of: an eccentric rotating mass (ERM) motor, a linear resonant actuator (LRA), a piezoelectric motor, a haptic motor/engine, or any other form of vibration device described herein. A battery 1015 (FIG. 10B) may be disposed within the housing 1020 for powering the transducer 1012 and the vibration device 1014 and control electronics that may be housed in the acoustic module 1002 and/or in the housing 1020. The control electronics can be used to control the vibration device 1014 and the transducer 1012.

Open audio device body 1016 can include curved bridge portion 1018, and housing 1020 with free distal end 1028. Bridge 1018 merges smoothly into acoustic module 1002, e.g., as shown in FIG. 10B, such that the beginning of the outer surface 1022 of bridge 1018 is tangent to the front curved portion of acoustic module 1002. Bridge 1018 is thinner than housing 1020. One reason is so that room is available for eyeglass temple pieces to still fit on the ear when a user is wearing the open audio device. In an example body 1016 is an integral molded plastic member. In an example body 1016 is made of another stiff material, such as metal. Body 1016 is in an example relatively stiff but may have some compliance in bridge portion 1018 as described below. Body 1016 is generally configured to be located behind the outer ear. Gap 1024 between body 1016 and acoustic module 1002 is generally sized and shaped to allow the upper portion of outer ear to fit through the opening, with the upper end 1026 of gap 1024 located such that the upper end of the helix is fitted in gap portion 1026. The upper end of the helix thus becomes a point about which open audio device 1000 can pivot or rotate.

Almost all of body 1016 sits behind the ear, along the intersection of the back of the ear and the head. Body 1016 is sized, shaped, contoured and angled relative to acoustic module 1002 such that body 1016 generally follows the shape and contour of the ear-head intersection and contacts the ear and/or head along much of the length of body 1016, almost to free distal end 1028. At the same time body 1016 slightly pushes the back of the outer ear out or away from the head. This bend of the ear causes a slight force against body 1016 that tends to push it against the head. Acoustic module 1002 has an inner face 1006 that is configured to sit against the front portion of outer ear (e.g., against one or more of fossa, anti-helix, crus of helix, and helix) as well as the portion of the head that is located immediately anteriorly of ear portion. The portion of acoustic module 1002 proximate the uppermost point 1030 of inside surface 1032 of body 1016 may sit under helix.

The head and the upper portion of the ear are stiffer than is the back of the outer ear. Since acoustic module 1002 is sitting against a hard surface it is not able to move closer to the head. This forces body 1016 to push out into outer ear, which creates an opposing force that tends to rotate open audio device 1000 about point 1030. This results in three constraining device anchoring locations, which include the device contacting the helix around point 1030, the acoustic module 1002 resting against the ear and head, and the body 1016 pushing toward the head due to the slightly bent soft part of the ear. The flexibility of the outer ear loads/preloads these three points to ensure they are always experiencing a normal force. The flexibility of the outer ear thus contributes to a stable yet comfortable fit of open audio device 1000. Also, since the three anchoring locations are not linear they generally define the apices of a triangle, which creates greater stability than if the anchor locations were aligned. Open audio device 1000 is thus gently but firmly held on the head, even when the head moves. Open audio devices that are configured to be carried on or proximate the ear in a similar manner are disclosed in U.S. patent application Ser. No. 62/952,873 filed on Dec. 23, 2019, the entire disclosure of which is incorporated herein by reference.

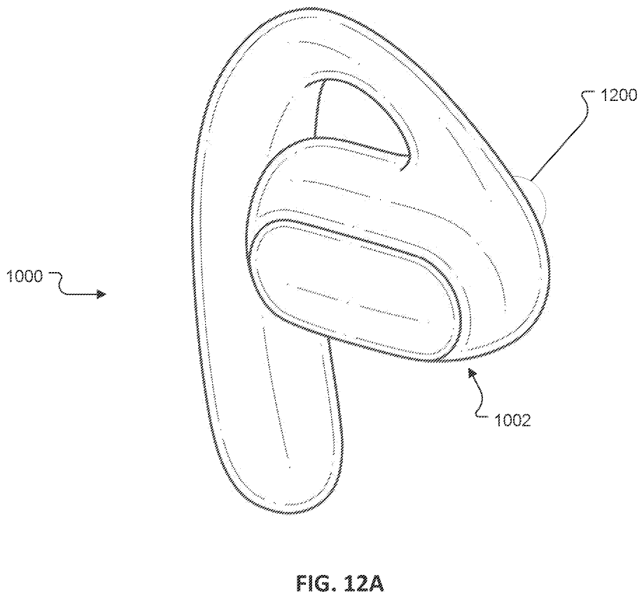

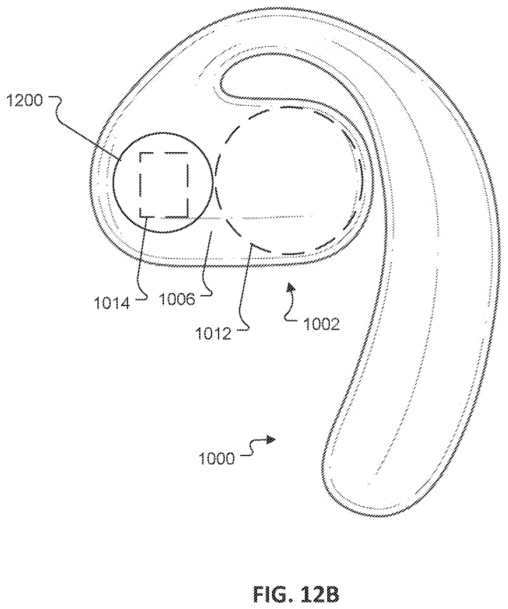

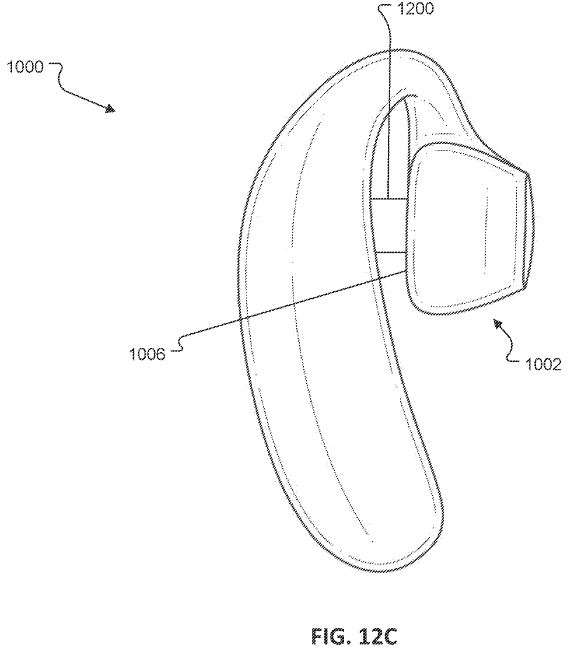

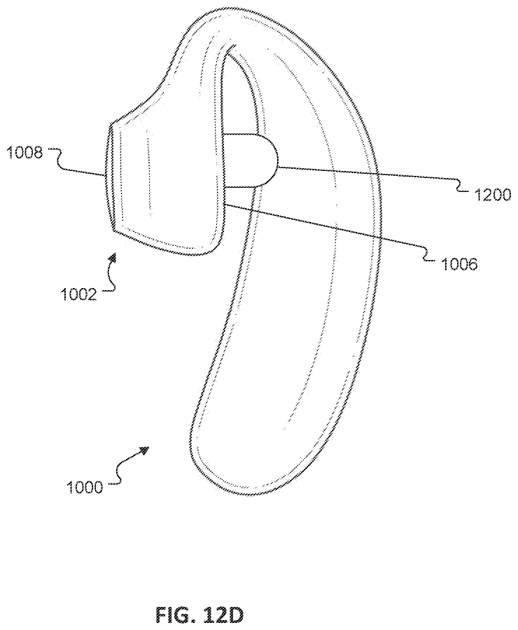

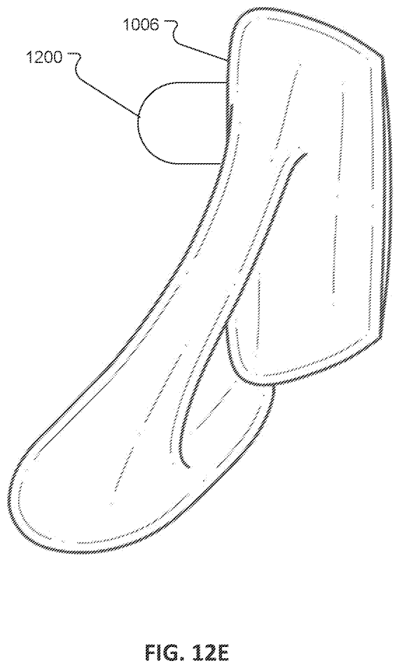

With reference to FIGS. 12A-12E, in some implementations, the open audio device 1000 may include a protrusion 1200 that extends outwardly from the inner face 1006 of the acoustic module 1002. The vibration device 1014 is supported in or coupled to the protrusion 1200 so as to vibrate the protrusion 1200. The protrusion 1200 is positioned such that it extends into the user's cymba concha when the device 1000 is worn; i.e., such that vibrotactile stimulation from the vibration device 1014 is transmitted to the user's cymba concha via the protrusion 1200.

In still other implementations, an extension member similar to protrusion 1200 in FIGS. 12A-12E can be integrated in an on-ear and/or over-ear audio device such as a pair of audio eyeglasses, on-ear headphones or over-ear headphones, and can be controlled to provide vibrotactile stimulation of one or more nerves proximate a user's ear. In some cases, the extension member is positioned proximate the concha region and/or ear canal of the user when the earpiece is positioned on or over the ear of the user.

As noted herein, in various implementations, the earpieces 100, 600, 700, 800, 900, 1000 in wearable devices can include an additional vibration device, e.g., in each earpiece. That is, one or more of the earpieces in a given set of earpieces can include two or more vibration devices. In certain implementations, vibration devices in a given earpiece 100, 600, 700, 800, 900, 1000 are of the same type (e.g., two ERM motors, two LRAs, two piezoelectric motors, etc.). In other implementations, a given earpiece 100, 600, 700, 800, 900, 1000 can include two distinct types of vibration device (e.g., an ERM motor and an LRA, or a piezoelectric motor and a focused ultrasound device). In certain cases, a set of two earpieces includes at least one earpiece with two or more vibration devices.

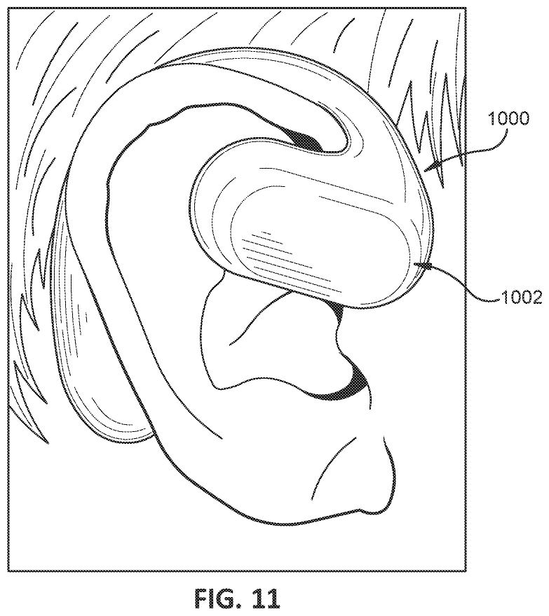

In further implementations, one or more of the earpieces 100, 600, 700, 800, 900, 1000 in wearable devices described herein includes a thermal actuator 150, illustrated in phantom in various depictions in FIGS. 1-12 (e.g., FIG. 1A, FIG. 4, FIG. 6A, FIG. 7A, FIG. 7D, FIG. 8, FIG. 9, FIG. 10B, FIG. 10C) as optional. In particular cases, the thermal actuator 150 is located proximate the concha region and/or the ear canal of the user. The thermal actuator 150 can include a heating and/or cooling element. In particular examples, the thermal actuator can include a heating element such as a direct resistance electrical heating element, e.g., with resistance coming from any suitable electrically conductive material including metals, conductive fluids, conductive polymers and/or other resistance heating materials such as mica. In particular cases, the heating element includes a heating coil. In other cases, the heating element includes a Peltier device that includes a solid-state heat pump and can be used to deliver heat. A cooling element can also include a Peltier device (e.g., a thermoelectric device), which in some cases, can be used for heating and/or cooling. A cooling element can also include a thermo-acoustic engine, which utilizes sound to induce temperature change.

The thermal actuator 150 can be connected with the controller 300 (FIG. 3), and can be actuated according to a modulated pattern to stimulate at least one nerve proximate the user's ear. In certain cases, the thermal actuator 150 supplements the vibration device(s) described herein, enabling stimulation of the user's ear region with both vibrotactile stimulus and thermal stimulus (e.g., heat and/or cold).

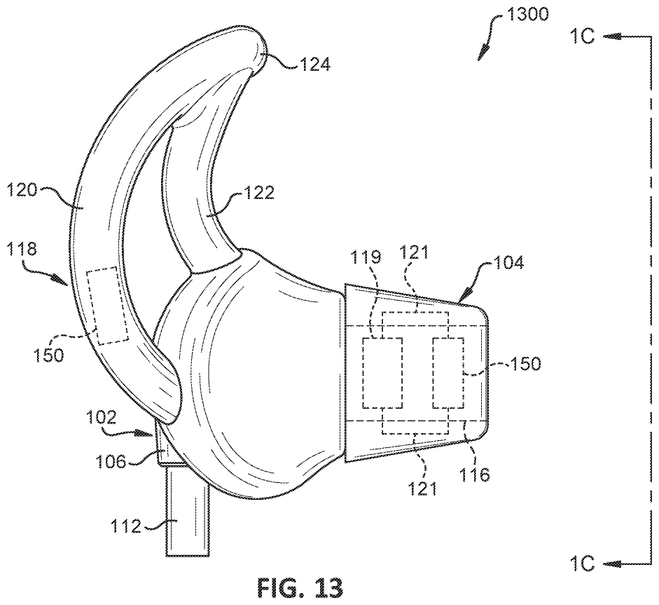

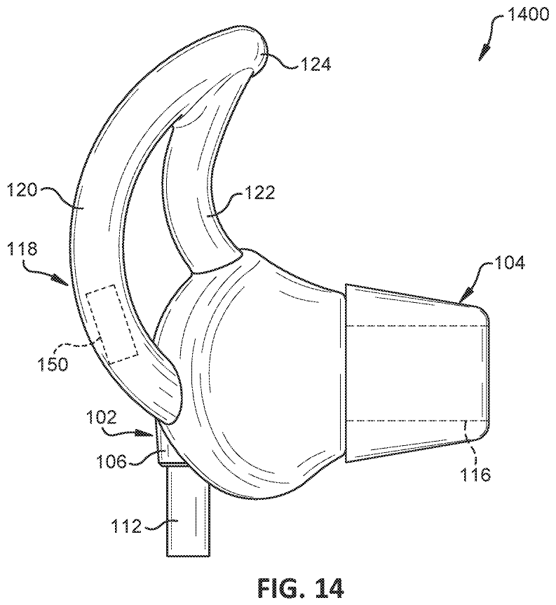

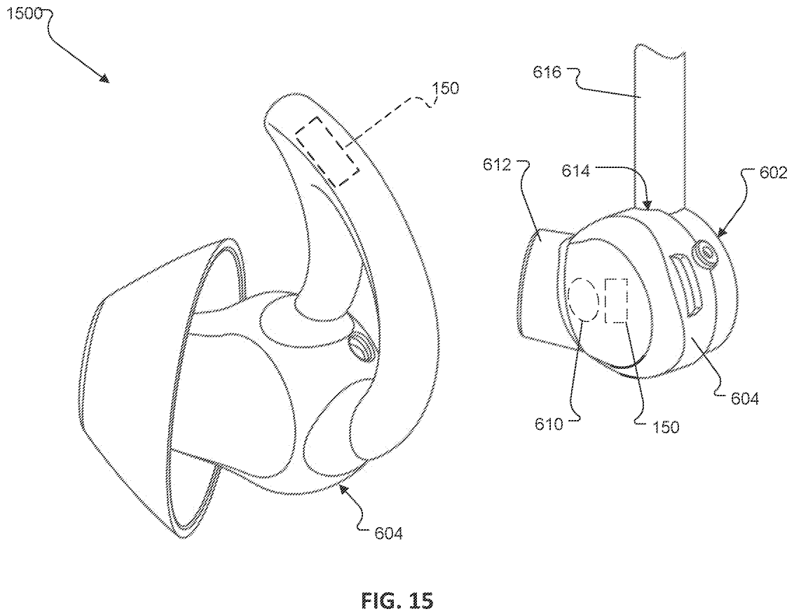

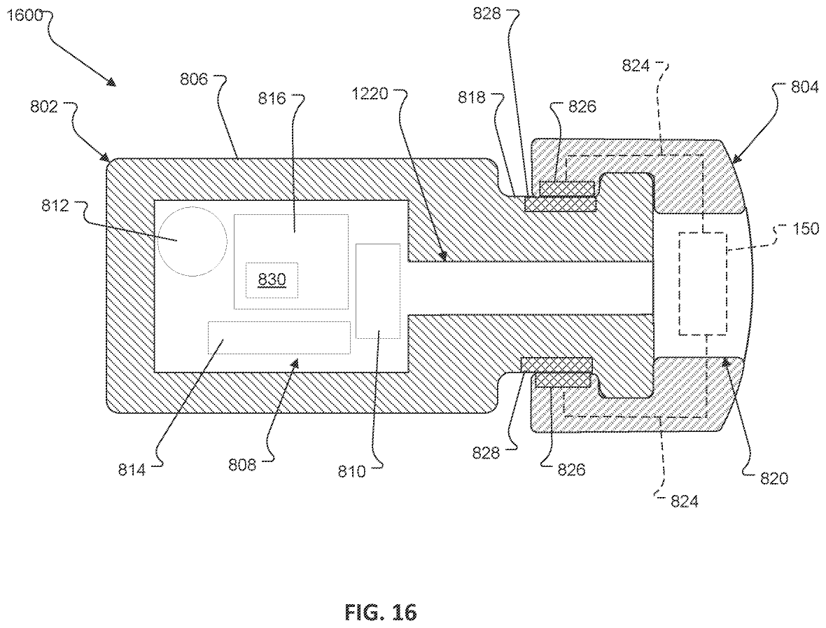

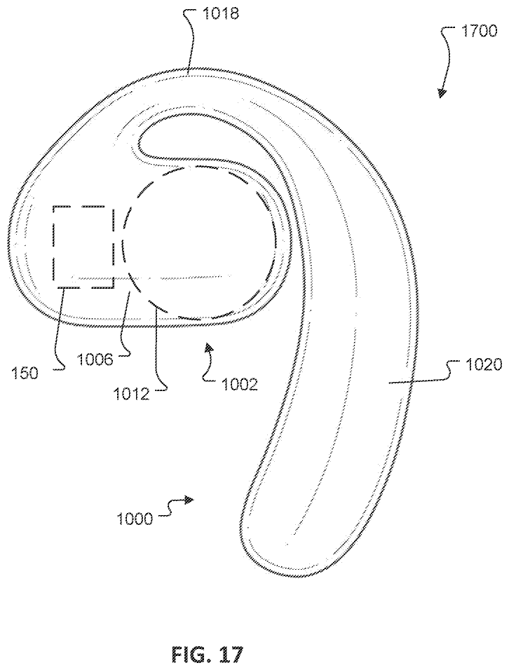

In additional implementations, as illustrated in the earpieces 1300, 1400, 1500, 1600, 1700 in FIGS. 13, 14, 15, 16 and 17 (as examples), the thermal actuator 150 functions as the only stimulating device in one or both earpieces. In these cases, the controller 300 (FIG. 3) is configured control the thermal actuator 150 to stimulate one or more nerves proximate the user's ear. In particular aspects, the controller 300 (FIG. 3) initiates thermal stimulation of the nerve(s) using one or more modulated patterns described herein. In some such cases, the modulated pattern is applied using only thermal stimulus, and without vibrotactile stimulus described with respect to FIGS. 1-12.

In still further implementations, one or more of the earpieces 100, 600, 700, 800, 900, 1000 in wearable devices described herein includes a tactile actuator 160, illustrated in phantom in various depictions in FIGS. 1-12 (e.g., FIG. 1A, FIG. 1C, FIG. 1D, FIG. 10B) as optional. In these implementations, the tactile actuator 160 can supplement the vibration device(s) described herein to provide a low-frequency tactile stimulus to the nerve(s) proximate the ear of the user. In certain cases, the controller 300 (FIG. 3) actuates the tactile actuator 160 to provide the low-frequency tactile stimulus characterized by a frequency of approximately ½ Hertz (Hz) or less.

Stimulation Approaches

In particular implementations, the controller 300 (FIG. 3) described herein is configured to actuate the vibration device (e.g., vibration devices 117, 608, 822, 920, 1014) according to a modulated pattern to stimulate at least one nerve proximate the user's ear (e.g., vagus nerve, trigeminal nerve, and/or facial nerve). In various implementations, a nerve stimulation program (e.g., program code or other instructions stored in memory 302 and/or mass storage device 310) is executed by the processor in controller 300 to initiate one or more approaches for nerve stimulation with a modulated pattern. In certain cases, the modulated pattern is characterized by: i) a peak drive level sufficient to stimulate nerve endings of at least one never proximate the user's ear; and ii) a carrier frequency (or, fundamental frequency) of approximately 50 Hertz (Hz) to approximately 500 Hz. In particular cases, the carrier frequency is equal to approximately 200 Hz.

In some examples, the peak drive level applies a force to the concha region and/or the ear canal of the user of approximately 0.1 Newton (N) to approximately 0.9 N., and in particular cases, approximately 0.3 N to approximately 0.7 N, and in more particular cases, approximately N.

In various implementations, the modulated pattern is characterized, in particular, by variation in the peak drive level (amplitude) of the stimulation. That is, in certain cases, the pattern's modulation is caused primarily by a change in the peak drive level of the stimulation. It is understood that in these cases, the carrier frequency of the stimulation is also adjusted, but that carrier frequency adjustment can be caused, at least in part, by the adjustment in peak drive level.

Additionally, as described herein, the controller 300 can be configured to adjust the modulated pattern at the vibration device by adjusting the peak drive level (amplitude) and/or the carrier frequency. That is, the controller 300 is configured in various implementations to adjust the modulated pattern applied by the vibration device by adjusting the peak drive level (amplitude) of stimulation, the carrier frequency of stimulation, or both the peak drive level and the frequency of stimulation.

In certain cases, the vibration device (e.g., vibration device(s) 117, 608, 822, 920, 1014) vibrates at the modulated carrier frequency and force in response to a stimulation waveform controlled by the controller 300 (FIG. 3). In various implementations, the modulated pattern is further characterized by one of: a) a fast onset and a gradual decay, b) a fast onset, a gradual decay, and a period of nominal vibrational force, c) a fast onset, a gradual decay, and a period of zero vibrational force, d) a symmetric onset and decay, or e) a multi-frequency pattern including a first segment with a first frequency and a first period, and a second segment with a second, higher frequency and a second, longer period. In certain implementations with at least a nominal vibrational force between peak modulations, users may experience less jarring than implementations with a zero vibrational force between peak modulations.

In certain cases, the modulated pattern approximates a breath rate of the user, e.g., to enhance particular health benefits. For example, approximating the breath rate of the user can aid in breathing entrainment, which in turn can help with relaxation and stress reduction, etc. In some cases, entrainment of stimulation to breath rate has the potential to amplify the naturally occurring increase in parasympathetic tone during exhalation. In particular implementations, the modulated pattern has a period equal to approximately one second to approximately 20 seconds, with a particular example period equal to approximately 7 seconds.

In certain examples, the modulated pattern includes a session of recurring modulated patterns that last up to approximately 20 minutes. In these cases, shorter modulated patterns (e.g., of several minutes or less) are repeated several times in a given session. According to some implementations, the sessions are repeated two or more times in a day, e.g., three, four, five times or more. In certain implementations, sessions are initiated at particular times of the day, or days of the week, e.g., to enhance health benefits. For example, sessions are initiated during the evening prior to sleep in some cases. In other cases, sessions are initiated during sleep, e.g., where the earpiece is part of a sleep aid device. In still further cases, sessions are initiated on particular days of the week and/or times of the day, e.g., on Monday or Tuesday mornings to mitigate work-related stress, or on Friday evenings to enhance winding down from the work week.

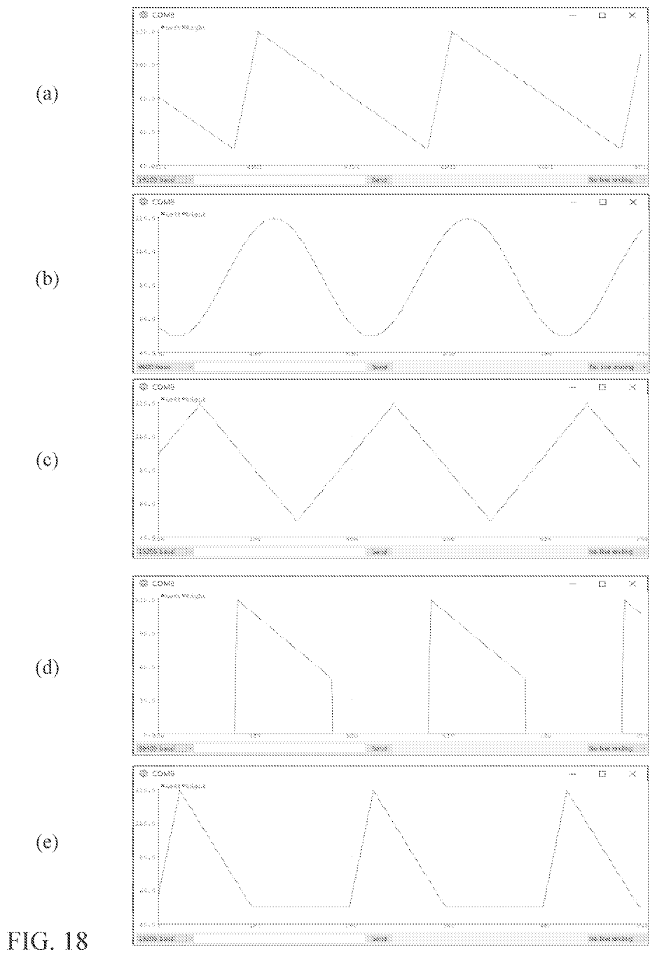

In certain cases, the controller 300 (FIG. 3) is configured to initiate the modulated pattern of stimulus in only one earpiece at a given time, and in some cases, in only one earpiece in a session. Example waveforms for driving the vibration device (e.g., vibration device(s) 117, 608, 822, 920, 1014) in a single earpiece are illustrated in the graphs (a)-(d) in FIG. 18. In these examples, waveforms (a)-(d) represent stimulation of the right-ear earpiece, but can equally apply to a left-ear earpiece. The waveforms modulate the amplitude, and to a lesser degree, the frequency of the carrier vibration that is delivered. Modulating the waveforms in this way can create a pleasing experience for the user while imparting enough vibrational force to stimulate the surrounding nerves.

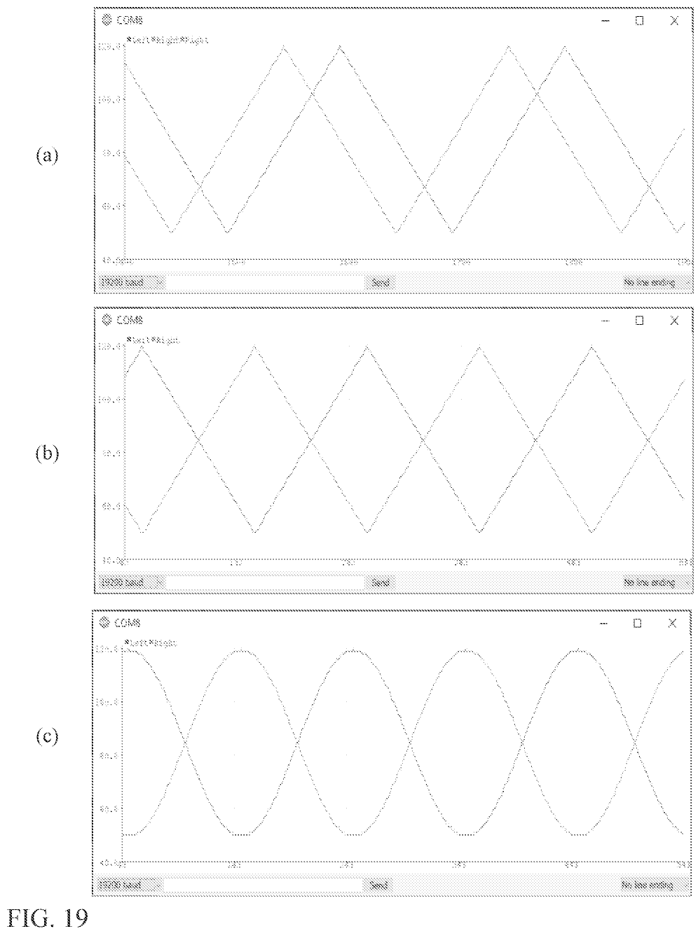

In additional implementations, the controller 300 (FIG. 3) is configured to initiate bilateral stimulation of the user using a modulated pattern at both earpieces in a wearable device. Example waveforms for driving a vibration device (e.g., vibration device(s) 117, 608, 822, 920, 1014) in two earpieces are illustrated in the graphs (a)-(c) in FIG. 19. In various particular implementations, as illustrated in graphs (a)-(c) in FIG. 19, the modulated pattern can further be characterized by a phase difference between the user's ears, illustrated by left ear (top line) and right ear (bottom line). In certain cases, the phase difference is equal to 180 degrees, e.g., as shown in graphs (b) and (c). In other cases, the phase difference is less than 180 degrees, e.g., as shown in graph (a). In still other implementations, the left and right earpieces deliver stimulus in phase with one another (i.e., phase difference of zero). A phase difference can create a pleasing experience for the user.

In still further, implementations, the controller 300 (FIG. 3) is configured to adjust the modulation pattern (e.g., in terms of amplitude (peak drive level) and/or frequency) in response to receiving biometric feedback about the user from a sensor system. For example, a sensor system located within the earpiece(s) 100, 600, 700, 800, 900, 1000, 1300, 1400, 1500, 1600, 1700 or connected with the earpiece(s) 100, 600, 70, 800, 900, 1000, 1300, 1400, 1500, 1600, 1700 (e.g., in a connected control device or hub, a connected smart device, a biometric tracker such as a fitness band, watch, ring, etc., or another device configured to provide biometric feedback) can indicate a biometric characteristic of the user, and based on that indicated biometric characteristic, the controller 300 (FIG. 3) is configured to adjust the modulation of the stimulation pattern. For example, the sensor system can include one or more monitors for detecting at least one of: breath rate, heart rate, body temperature, etc. In various implementations, the controller 300 (FIG. 3) compares the detected biometric feedback with one or more biometric thresholds to determine whether the user is in a particular biometric state (e.g., exercising, stressed, relaxed, etc.). In some cases, biometric thresholds are saved in the memory 302 (FIG. 3), or are otherwise accessible by the controller 300 (FIG. 3) for use in evaluating biometric feedback. In certain cases, biometric thresholds are user-specific, e.g., developed using a model of the user's biometric activity over a period. In other cases, biometric thresholds are generalized for a particular group of users (e.g., based on one or more physical characteristics). In some examples, the detected biometric feedback can indicate that the user is in one or more biometric states, e.g., exercise state, stressed state, cool-down state, relaxed state, etc.

According to various implementations, the vibration applied proximate the user's ear can include applying pressure such as a tap, bump, pulse, etc. in any pattern described herein. In some cases, static application of pressure (e.g., with tactile actuator 160) is used to supplement the vibrotactile and/or thermal stimulation approaches described herein.

In certain of these cases, vibration is applied to one or more regions of the head of the user, e.g., to the temple region(s), the bridge of the nose, the ear region(s), the scalp, the nape, and/or the neck of the user. In some cases, the controller 300 is configured to vary the vibration pattern based on the location of the application on the user's head. According to particular implementations, distinct forms of wearable device are configured to apply vibration to different regions of the head of the user, e.g., such that an over-ear headphone can apply vibration to one or more regions proximate the user's ears (e.g., temple region) and/or proximate a headband, while an in-ear headphone such as an earbud can apply pressure in a more localized region of the user's ears, and audio eyeglasses can apply pressure in regions proximate the user's ears as well as across the bridge of the nose, the temple region, etc. In one particular example, the controller 300 is configured to initiate vibration at a wearable device (e.g., at earpiece(s) 100, 600, 700, 800, 900, 1000, 1300, 1400, 1500, 1600, 1700) in particular, to apply vibration to the ear region of the user proximate the ear canal entrance or the cymba concha. In these examples, application of vibration proximate the ear canal entrance or the cymba concha can be beneficial because the vagus nerve innervates those regions. Stimulating the vagus nerve can induce neural plasticity.

In particular cases where the earpieces 1300, 1400, 1500, 1600 (FIGS. 13-16) include a thermal actuator 150 for applying a thermal stimulus proximate the user's concha region and/or the ear canal, the controller 300 (FIG. 3) is configured to stimulate at least one of the nerves proximate the ear with the thermal actuator 150 according to a modulated pattern. In certain cases, the modulated pattern relying on thermal stimulation is characterized by: i) a peak thermal differential sufficient to stimulate nerve endings of the nerve(s) proximate the ear of the user; and ii) a frequency of 0 Hertz (Hz) (i.e., constant thermal stimulation) to approximately 1 Hertz (Hz) (and in some cases, approximately 0.1 Hz to approximately 1 Hz). In some cases, the peak thermal differential is equal to approximately 0.5 degrees Celsius (C) to approximately 15 degrees C. In certain cases, the peak thermal differential is equal to approximately +15 degrees C. from the user's body temperature to approximately −37 degrees C. from the user's body temperature (e.g., assuming body temperature at or near 98.6 degrees C.). The modulated pattern can be repeated in sessions similar to the modulated pattern of vibrotactile stimulation described herein, and can aid in mitigating and/or treating any health condition described herein.

As noted herein, the controller 300 (FIG. 3) can instruct the transducer 114 (FIG. 1D) to provide an audio output in addition to the vibrotactile and/or thermal stimulus, e.g. an audio noise signal configured to entrain the user's breathing (e.g., any noise signal such as one or more tone bursts, chirps, etc.), or a noise signal (e.g., white noise and/or pink noise). In various implementations, the controller 300 is configured to aid in tinnitus therapy by initiating the audio output (e.g., at transducer(s) 114) as: a) broadband sounds with a notch at a frequency range approximating a frequency at which the user perceives the tinnitus; b) broadband sounds with a peak at a frequency range approximating a frequency at which the user perceives the tinnitus; c) at least one tone (e.g., a soft tone) at a frequency outside of the frequency range approximating the frequency at which the user perceives the tinnitus; and/or d) at least one tone at the frequency range approximating the frequency at which the user perceives the tinnitus (e.g., as detected by feedback from the user). The term “approximately” as used with respect to values herein can allot for a nominal variation from absolute values, e.g., of several percent or less.

In any case, the wearable devices shown and described herein are configured to aid in treating and/or managing one or more health conditions for a user, especially those that use relaxation to mitigate the condition, e.g., stress, depression, pain, anxiety, tinnitus, sleep disorders, etc. In other cases, the wearable devices shown and described herein are configured to provide additional health and wellness benefits, particularly for conditions where relaxation is beneficial such as stress-related and/or stress-induced conditions. Stress, depression, pain and sleeping disorders are all health concerns with high incidence in the general population and are affected by sympathetic/parasympathetic balance. Stimulation of peripheral nerves at the ear can increase parasympathetic tone and thus positively impact the conditions noted herein. Further, tinnitus can be affected because the auditory system integrates sound with somatic information from peripheral nerves such as the auricular branch of the vagus nerve.

In addition to the above-noted benefits, the wearable devices shown and described herein can be easily adapted for frequent use and/or use in different conditions, which can help aid in compliance. As such, the wearable devices disclosed according to various implementations can improve user experiences as well as health outcomes when compared with conventional nerve stimulation approaches and related systems.

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as, special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

Elements of figures are shown and described as discrete elements in a block diagram. These may be implemented as one or more of analog circuitry or digital circuitry. Alternatively, or additionally, they may be implemented with one or more microprocessors executing software instructions. The software instructions can include digital signal processing instructions. Operations may be performed by analog circuitry or by a microprocessor executing software that performs the equivalent of the analog operation. Signal lines may be implemented as discrete analog or digital signal lines, as a discrete digital signal line with appropriate signal processing that is able to process separate signals, and/or as elements of a wireless communication system.

When processes are represented or implied in the block diagram, the steps may be performed by one element or a plurality of elements. The steps may be performed together or at different times. The elements that perform the activities may be physically the same or proximate one another, or may be physically separate. One element may perform the actions of more than one block. Audio signals may be encoded or not, and may be transmitted in either digital or analog form. Conventional audio signal processing equipment and operations are in some cases omitted from the drawings.

In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

Other embodiments not specifically described herein are also within the scope of the following claims. Elements of different implementations described herein may be combined to form other embodiments not specifically set forth above. Elements may be left out of the structures described herein without adversely affecting their operation. Furthermore, various separate elements may be combined into one or more individual elements to perform the functions described herein.

1. A wearable device for providing vibrotactile stimulus to at least one nerve proximate an ear of a user, the device comprising:
an earpiece comprising a vibration device for application of vibrotactile stimulus proximate at least one of a concha region or an ear canal of the user; and
a controller connected with the vibration device, the controller configured to actuate the vibration device according to a modulated pattern to stimulate at least one nerve proximate the ear of the user, wherein the modulated pattern is characterized by:
a peak drive level sufficient to stimulate nerve endings of at least one nerve proximate the ear of the user; and
a carrier frequency of approximately 50 Hertz (Hz) to approximately 500 Hz.

2. The wearable device of claim 1, wherein the peak drive level applies a force to at least one of the concha region or the ear canal of the user of approximately: i) 0.1 Newton (N) to approximately 0.9 N, approximately: ii) 0.3 N to approximately 0.7 N, or iii) approximately 0.5 N.

3. (canceled)

4. (canceled)

5. The wearable device of claim 1, wherein the controller is configured to adjust the modulated pattern in response to receiving biometric feedback about the user from a sensor system.

6. The wearable device of claim 1, wherein the vibration device vibrates at the carrier frequency, and the modulated pattern is controlled by a stimulation waveform, wherein the modulated pattern is further characterized by one of:
a) a fast onset and a gradual decay,
b) a fast onset, a gradual decay, and a period of nominal vibrational force,
c) a fast onset, a gradual decay, and a period of zero vibrational force,
d) a symmetric onset and decay, or
e) a multi-frequency pattern comprising a first segment with a first frequency and a first period, and a second segment with a second, higher frequency and a second, longer period.

7. The wearable device of claim 1, wherein the modulated pattern approximates a breath rate of the user and has a period equal to approximately one second to approximately 20 seconds.

8. (canceled)

9. The wearable device of claim 1, further comprising:
an electro-acoustic transducer connected with the controller and configured to provide an audio output, wherein the audio output comprises: an audio noise signal configured to entrain the user's breathing, or a noise signal.

10. (canceled)

11. The wearable device of claim 1, further comprising:
a thermal actuator connected with the controller for thermally stimulating an area proximate the ear of the user.

12. The wearable device of claim 1, wherein the wearable device comprises an in-ear audio device, an on-ear audio device or an over-ear audio device, wherein, in the case that the wearable audio device comprises an on-ear audio device or an over-ear audio device, the earpiece further comprises an extension member for positioning proximate at least one of the concha region or the ear canal of the user when the earpiece is positioned on or over the ear of the user.

13. (canceled)

14. The wearable device of claim 1, further comprising:
an additional earpiece comprising an additional vibration device for application of vibrotactile stimulus proximate at least one of a concha region or an ear canal of an additional ear of the user,
wherein the controller is connected with the additional vibration device and is configured to actuate the vibration device and the additional vibration device according to the modulated pattern,
wherein the modulated pattern is further characterized by a phase difference between the user's ears, and wherein the phase difference is equal to or less than 180 degrees.

15-17. (canceled)

18. The wearable device of claim 1, wherein the earpiece comprises an eartip and a support structure extending from the eartip, wherein the vibration device is located in the support structure.

19. (canceled)

20. The wearable device of claim 1, wherein the vibration device comprises at least one of: an eccentric rotating mass (ERM) motor, a linear resonant actuator (LRA), a piezoelectric motor, a haptic engine, a focused ultrasound device, or a transducer without a voice coil, and wherein the earpiece is in electrically non-conducting contact with the user's skin during use.

21. The wearable device of claim 1, wherein the controller comprises a control circuit contained in the earpiece, wherein the control circuit is configured to receive commands from at least one of a local logic engine or a remote logic engine for actuating the vibration device according to the modulated pattern.

22. (canceled)

23. The wearable device of claim 1, wherein the modulated pattern comprises a session of recurring modulated patterns lasting approximately 15-20 minutes or less wherein the session is repeated at least two times throughout a day.

24. (canceled)

25. The wearable device of claim 1, further comprising a tactile actuator coupled with the controller for providing a low-frequency tactile stimulus to the at least one nerve proximate the ear of the user, wherein the low-frequency tactile stimulus is characterized by a frequency of approximately ½ Hertz (Hz) or less.

26. The wearable device of claim 1, further comprising: an additional vibration device in the earpiece and coupled with the controller, wherein the controller is configured to actuate the additional vibration device according to the modulated pattern.

27. A wearable device for providing vibrotactile stimulus to at least one nerve proximate an ear of a user, the device comprising:
a set of two earpieces each comprising a vibration device for application of vibrotactile stimulus proximate at least one of a concha region or an ear canal of the user's ears; and
a controller connected with the vibration devices, the controller configured to actuate the vibration devices according to a modulated pattern to stimulate at least one nerve proximate the ear of the user, wherein the modulated pattern is characterized by:
a peak drive level sufficient to stimulate nerve endings of the at least one nerve proximate the ear of the user;
a carrier frequency of approximately 50 Hertz (Hz) to approximately 500 Hz; and
a phase difference between the modulated pattern at the vibration devices,
wherein the peak drive level applies a force to at least one of the concha region or the ear canal of the user of approximately 0.3 N to approximately 0.7 N.

28. The wearable device of claim 27, wherein the phase difference is equal to or less than 180 degrees.

29. (canceled)

30. (canceled)

31. The wearable device of claim 27, wherein the modulated pattern is further characterized by one of:
a) a fast onset and a gradual decay,
b) a fast onset, a gradual decay, and a period of nominal vibrational force,
c) a fast onset, a gradual decay, and a period of zero vibrational force,
d) a symmetric onset and decay, or
f) a multi-frequency pattern comprising a first segment with a first frequency and a first period and a second segment with a second, higher frequency and a second, longer period.

32. The wearable device of claim 27, wherein the modulated pattern either:
i) approximates a breath rate of the user and has a period equal to approximately one second to approximately 20 seconds, wherein the period is equal to approximately 7 seconds, or
ii) comprises a session of recurring modulated patterns lasting approximately 15-20 minutes or less, wherein the session is repeated at least two times throughout a day.

33-43. (canceled)

44. The wearable device of claim 27, wherein at least one of the earpieces further comprises a tactile actuator connected with the controller for providing a low-frequency tactile stimulus to the at least one nerve proximate the ear of the user, wherein the low-frequency tactile stimulus is characterized by a frequency of approximately ½ Hertz (Hz) or less.

45-47. (canceled)
